(19) United States
(12) Patent Application Publication
(10) Pub. No.: US 2026/0021200 A1
(43) Pub. Date: Jan. 22, 2026

(54) MUSCLE-SPECIFIC REGULATORY CASSETTES

(71) Applicants: NEVADA RESEARCH & INNOVATION CORPORATION, Reno, NV (US); BOARD OF REGENTS OF THE NEVADA SYSTEM OF HIGHER EDUCATION, ON BEHALF OF THE UNIVERSITY OF NEVADA, Reno, NV (US)

(72) Inventors: Peter L. Jones, Reno, NV (US); Charis L. Himeda, Reno, NV (US)

(21) Appl. No.: 18/996,538

(22) PCT Filed: Jul. 19, 2023
(86) PCT No.: PCT/US2023/070498

§ 371 Date: Jan. 17, 2025

Related U.S. Application Data
(60) Provisional application No. 63/390,886, filed on Jul. 20, 2022.

(51) Int. Cl. A61K 48/00; C07K 14/005; C12N 15/86

(52) U.S. Cl. CPC A61K 48/0041; C07K 14/005; C12N 15/86; C12N 2750/14122; C12N 2750/14143; C12N 2750/14145

(57) ABSTRACT

The present disclosure relates to nucleic acid regulatory cassettes useful for the expression of transgene payloads in specific muscle tissues. Also included in the present disclosure are vectors, including adeno-associated virus (AAV) vectors, comprising the regulatory cassettes of the invention, as well as compositions and methods of treating genetic diseases or disorders also comprising said regulatory cassettes.

### CROSS-REFERENCE TO RELATED APPLICATIONS

The present application claims priority under 35 U.S.C. § 119 (e) to U.S. Provisional Patent Application No. 63/390,886, filed Jul. 20, 2022, which is hereby incorporated by reference in its entirety herein.

### SEQUENCE LISTING

The instant application contains a Sequence Listing which has been submitted in XML format via Patent Center and is hereby incorporated by reference in its entirety. Said XML file, created on Jul. 19, 2023, is named “369055_7028WO1_00100_SequenceListingST_26.xml” and is 4,096 bytes in size.

### BACKGROUND OF THE INVENTION

AAV-mediated gene therapies for muscle disorders require regulatory cassettes that provide high-level, striated muscle-specific activity. However, cardiotoxicity caused by transgene expression in the heart has emerged as a serious concern in clinical trials for Duchenne muscular dystrophy and X-linked myotubular myopathy. Additionally, the size of some therapeutic cargo requires regulatory cassettes to be even smaller than current, established versions. Thus there is a clear need in the art for highly minimized regulatory cassettes that retain high activity in skeletal muscles with either low or no activity in cardiac muscle. The present invention addresses this need.

### SUMMARY OF THE INVENTION

As described herein, the present disclosure relates to nucleic acids encoding regulatory cassettes useful for the expression of transgene payloads in specific muscle tissues. Also included are vectors, including AAV vectors, comprising the regulatory cassettes of the invention, as well as compositions and methods of treating genetic diseases or disorders also comprising said regulatory cassettes.

As such, in one aspect, the invention includes a polynucleotide encoding a muscle-specific regulatory cassette comprising at least three modified enhancer sequences upstream and operably linked to a promoter, wherein the three modified enhancer sequences comprise a Trex sequence, an AT-rich sequence, a right E-box sequence, and a MEF2 sequence, wherein the right E-box sequence is repeated at least once, and wherein the promoter is a −80 to +50 promoter containing a consensus Inr sequence.

In certain embodiments, the cassette has high expression activity in skeletal muscle tissue.

In certain embodiments, the skeletal muscle tissue is fast-twitch muscle tissue.

In certain embodiments, the cassette has very low expression in cardiac tissue as compared to skeletal muscle tissue.

In certain preferred embodiments, the cassette is encoded by a nucleic acid comprising the sequence set forth in SEQ ID NO: 1.

In certain embodiments, the cassette is encoded by a nucleic acid comprising at least 80%, at least 85%, at least 90%, at least 95%, at least 98%, and at least 99% sequence identity to the sequence set forth in SEQ ID NO: 1.

In another aspect, the invention includes a polynucleotide encoding a muscle-specific regulatory cassette comprising at least three modified enhancer sequences upstream and operably linked to a promoter, wherein the at least three modified enhancer sequences comprise a Trex sequence, an AT-rich sequence, a left E-box sequence, a right E-box sequence, and a MEF2 sequence, wherein the promoter is a −80 to +50 promoter containing a consensus Inr sequence.

In certain embodiments, the cassette has high expression activity in skeletal muscle tissue.

In certain embodiments, the cassette has low expression activity in cardiac tissue compared to skeletal muscle tissue.

In certain preferred embodiments, the cassette is encoded by a nucleic acid comprising the sequence set forth in SEQ ID NO: 2.

In certain embodiments, the cassette is encoded by a nucleic acid comprising at least 80%, at least 85%, at least 90%, at least 95%, at least 98%, and at least 99% sequence identity to the sequence set forth in SEQ ID NO: 2.

In another aspect, the invention includes an AAV vector comprising a muscle-specific regulatory cassette comprising the polynucleotide of any one of the above aspects for embodiments or any other aspect or embodiment disclosed herein.

In another aspect, the invention includes an AAV vector comprising a muscle-specific regulatory cassette comprising the polynucleotide of any one of any one of the above aspects for embodiments or any other aspect or embodiment disclosed herein.

In certain embodiments, the AAV vector comprises an AAV capsid that is a serotype selected from the group consisting of AAV1, AAV2, AAV3, AAV3B, AAV4, AAV5, AAV6, AAV7, AAV8, AAV9, AAV12, AAV-B1, AAV-DJ, AAV-Retro, AAVrh8, AAVrh10, AAVrh25, Anc80L65, LK03, AAVrh18, rAAVrh74, AAVrh32.33, AAVrh39, AAVrh43, MyoAAV2, Oligo001, PHP-B, and Spark 100.

In certain preferred embodiments, the capsid is an rAAVrh74 serotype.

In certain preferred embodiments, the capsid is an AAV9 serotype.

In certain preferred embodiments, the capsid is a MyoAAV2 serotype.

In certain embodiments, the capsid has a specificity for muscle tissue.

In another aspect, the invention includes a composition comprising AAV vector particles comprising the polynucleotide of any one of the above aspects or embodiments or any aspect or embodiment disclosed herein and a pharmaceutically acceptable carrier or excipient.

In another aspect, the invention includes a method of treating a genetic disease or disorder in a subject in need thereof, comprising administering to the subject an effective amount of an AAV vector comprising a muscle-specific regulatory cassette comprising at least three modified enhancer sequences upstream and operably linked to a promoter, thereby treating the genetic disease or disorder, wherein the three modified enhancer sequences comprise a Trex sequence, an AT-rich sequence, a right E-box sequence, and a MEF2 sequence, wherein the right E-box sequence is repeated at least once, and wherein the promoter is a −80 to +50 promoter containing a consensus Inr sequence.

In certain preferred embodiments, the cassette is encoded by a nucleic acid comprising the sequence set forth in SEQ ID NO: 1.

In another aspect, the invention includes a method of treating a genetic disease or disorder in a subject in need thereof, comprising administering to the subject an effective amount of an AAV vector comprising a muscle-specific regulatory cassette comprising at least three modified enhancer sequences upstream and operably linked to a promoter, wherein the at least three modified enhancer sequences comprise a Trex sequence, an AT-rich sequence, a left E-box sequence, a right E-box sequence, and a MEF2 sequence, wherein the promoter is a −80 to +50 promoter containing a consensus Inr sequence.

In certain preferred embodiments, the cassette is encoded by a nucleic acid comprising the sequence set forth in SEQ ID NO: 2.

In certain embodiments, the AAV vector comprises an AAV capsid that is a serotype selected from the group consisting of AAV1, AAV2, AAV3, AAV3B, AAV4, AAV5, AAV6, AAV7, AAV8, AAV9, AAV12, AAV-B1, AAV-DJ, AAV-Retro, AAVrh8, AAVrh10, AAVrh25, Anc80L65, LK03, AAVrh18, rAAVrh74, AAVrh32.33, AAVrh39, AAVrh43, MyoAAV2, Oligo001, PHP-B, and Spark100.

In certain embodiments, the capsid is an rAAVrh74 serotype.

In certain embodiments, the capsid is an AAV9 serotype.

In certain embodiments, the capsid is a MyoAAV2 serotype.

In certain embodiments, the AAV vector has a specificity for muscle tissue.

In certain embodiments, the genetic disease or disorder is a muscle-related genetic disease or disorder.

In certain embodiments, the muscle-related genetic disease or disorder is selected from the list consisting of facioscapulohumeral muscular dystrophy (FSHD), X-linked myotubular myopathy (XLMTM), central core myopathy, inclusion body myositis, nemaline myopathy, distal myopathy, centronuclear myopathy, oculopharyngeal muscular dystrophy, a dysferlinopathy, a limb-girdle muscular dystrophy (LGMD), and Duchenne muscular dystrophy (DMD).

In another aspect, the invention includes a method of introducing a transgene into a target cell, comprising contacting the cell with a polynucleotide encoding a muscle-specific regulatory cassette comprising at least three modified enhancer sequences upstream and operably linked to a promoter, wherein the three modified enhancer sequences comprise a Trex sequence, an AT-rich sequence, a right E-box sequence, and a MEF2 sequence, wherein the right E-box sequence is repeated at least once, and wherein the promoter is a −80 to +50 promoter containing a consensus Inr sequence.

In certain embodiments, the cassette has high expression activity in skeletal muscle tissue.

In certain embodiments, the skeletal muscle tissue is fast-twitch muscle tissue.

In certain embodiments, the cassette has very low expression in cardiac tissue as compared to skeletal muscle tissue.

In certain preferred embodiments, The cassette is encoded by a nucleic acid comprising the sequence set forth in SEQ ID NO: 1.

In certain embodiments, the cassette is encoded by a nucleic acid comprising at least 80%, at least 85%, at least 90%, at least 95%, at least 98%, and at least 99% sequence identity to the sequence set forth in SEQ ID NO: 1.

In another aspect, the invention includes a method of introducing a transgene into a target cell, comprising contacting the cell with a polynucleotide encoding a muscle-specific regulatory cassette comprising at least three modified enhancer sequences upstream and operably linked to a promoter, wherein the at least three modified enhancer sequences comprise a Trex sequence, an AT-rich sequence, a left E-box sequence, a right E-box sequence, and a MEF2 sequence, wherein the promoter is a −80 to +50 promoter containing a consensus Inr sequence.

In certain embodiments, the cassette has high expression activity in skeletal muscle tissue.

In certain embodiments, the cassette has low expression activity in cardiac tissue compared to skeletal muscle tissue.

In certain preferred embodiments, the cassette is encoded by a nucleic acid comprising the sequence set forth in SEQ ID NO: 2.

In certain embodiments, the cassette is encoded by a nucleic acid comprising at least 80%, at least 85%, at least 90%, at least 95%, at least 98%, and at least 99% sequence identity to the sequence set forth in SEQ ID NO: 2.

### BRIEF DESCRIPTION OF THE DRAWINGS

The following detailed description of preferred embodiments of the invention will be better understood when read in conjunction with the appended drawings. For the purpose of illustrating the invention, there are shown in the drawings embodiments which are presently preferred. It should be understood, however, that the invention is not limited to the precise arrangements and instrumentalities of the embodiments shown in the drawings.

FIG. 1 depicts the evolution of the regulatory cassette platform. Illustrated is a schematic of tMCK and CK8 (original Ckm-based muscle regulatory cassettes) and the two new cassettes based on the CK8 design disclosed herein. The seven known transcriptional regulatory elements within the (′km enhancer are labeled. For CK8, changes from the endogenous sequence and/or previous (′km-based cassettes include: 1) deletion of 63 bp between the Right E-box and the MEF2 site; 2) 3 tandem copies of the modified enhancer; 3) mutation of sequence overlapping +1 to a consensus Initiator element (Inr); and 4) inclusion of +50 sequence downstream of the transcription start site. The NH cassette contains the following modifications from CK8: 1) CArG and AP2 sites removed; 2) sequence between regulatory elements minimized; 3) Left E-box mutated to a Right E-box; and 4) −80 basal promoter sequence used. The HLH cassette contains the following modification from the NH cassette: additional Right E-box replaced with the original Left E-box.

FIGS. 2A-2C depict the HLH cassette displaying higher activity than the NH cassette in skeletal muscle over time. The mCherry reporter under control of either the NH or HLH regulatory cassette was delivered in rAAVrh74 at 2.6×10¹⁴ GC/kg by retro-orbital injection to wild-type mice (n=3 per virus). The epi-fluorescent signal, measured as (FIG. 2A) total flux or (FIG. 2B) maximum radiance, in abdominal and pectoral muscles was (FIG. 2C) visualized at various times post-injection. Peak of Total flux and Max radiance of NH-mCherry signals are indicated with dashed line in FIG. 2A (1.27×10¹¹) and FIG. 2B (4.68×10⁹).

FIGS. 3A-3E depict the HLH cassette displaying higher activity than the NH cassette in all striated muscles. All mice from FIG. 2 were sacrificed at 12 weeks post-injection, individual tissues were dissected, and the epi-fluorescent signals were quantified as FIG. 3A) total flux or FIG. 3B) maximum radiance. FIG. 3B) Maximum radiance in each tissue was normalized to that in abdominal muscle from the same animal; for HLH-injected mice, signals from each tissue were further normalized to the average abdominal signal from NH-injected mice (set to 1). Representative images of isolated tissues are shown from FIG. 3C) uninjected mouse, FIG. 3D) NH-injected mouse, and FIG. 3E) HLH-injected mouse. Tissues in C and D are arranged as labeled in panel E. Abbreviations: tibialis anterior (TA), extensor digitorum longus (EDL), soleus (SOL), gastrocnemius (GA), quadriceps (QUA), abdominal muscles (Ab). Xs (panels A and B) indicate no signal above background detected.

FIGS. 4A-4F depict the HLH cassette displaying higher activity than the NH cassette in all striated muscles. Fluorescent signals in the isolated tissues from FIG. 3 were visualized with a 0.2-second exposure time. FIGS. 4A-4D, 4F) Expression of mCherry regulated by NH (left) and HLH (right) is compared to uninjected (CTL) tissues (middle). FIG. 4E) mCherry expression is compared between NH (left) and HLH (right). FIG. 4F) mCherry expression in soleus (three muscles in left sub-panel) and EDL (three muscles in right sub-panel). Abbreviations: tibialis anterior (TA), gastrocnemius (GA), quadriceps (QUA), soleus (SOL), extensor digitorum longus (EDL). Bar=2 mm.

FIGS. 5A-5B depict tissue transduction for rAAVrh74. The mCherry reporter under control of either FIG. 5A) NH or FIG. 5B) HLH was delivered systemically by retro-orbital injection to wild-type mice using rAAVrh74 virions at 2.6×10¹⁴ GC/kg. The rAAV genomes present in various tissues were assessed by qPCR using primers specific for the bGH PAS in the cargo genome and normalized to the single copy endogenous murine Rosa26 locus. This data confirms that tissues such as kidney and liver, which did not express any detectable mCherry signal by IVIS imaging, were still highly transduced by the virus, supporting the tissue specificity or tropism of the regulatory cassettes. Light grey bars indicate muscles with high mCherry expression; dark grey bars indicate muscles with low mCherry expression; black bars indicate muscles with undetectable mCherry expression. Abbreviations: tibialis anterior (TA), extensor digitorum longus (EDL), soleus (SOL), gastrocnemius (GA), quadriceps (QUA), abdominal muscle (Ab), diaphragm (Diaph).

FIGS. 6A-6L depict the NH cassette displaying high activity in most fast-twitch muscles and virtually no activity in EDL, soleus, diaphragm, and heart. The mCherry reporter under control of the NH regulatory cassette was delivered systemically by retro-orbital injection to wild-type mice using rAAVrh74 virions at 2.6×10¹⁴ GC/kg. The fluorescent signal was visualized at 12 weeks post-injection with the same exposure time of 1 second (1 s), except where indicated. Tissues dissected from rAAV-injected mice are indicated by asterisks and compared with tissues from uninjected mice. For two-tissue panels (FIGS. 6A-6B and 6D-6H), tissues from uninjected mice are shown on the left. For the four-tissue panel (FIG. 6C), uninjected tissues for soleus and EDL are on the left of each sub-panel. For single-tissue panels (FIG. 61-6L), panels J-L are rAAV-injected, while panel I is uninjected. Abbreviations: tibialis anterior (TA), gastrocnemius (GA), quadriceps (QUA), soleus (SOL), extensor digitorum longus (EDL).

FIGS. 7A-7J depict the NH cassette having no detectable activity in non-muscle tissues. Non-muscle tissues from the rAAV-transduced mice assayed in FIG. 6 were similarly imaged for mCherry expression (3-second exposure, except where indicated). rAAV-injected tissues are indicated by asterisks. For two-tissue panels (FIGS. 7B-7E and 7G-7J), tissues from uninjected mice are shown on the left. Single-tissue panels (FIGS. 7A and 7F) show only the tissues from rAAV-injected mice. FIG. 7A) The skeletal muscle from a hindlimb, including the posterior biceps femoris, quadriceps, and gastrocnemius, express mCherry, while the sciatic nerve, indicated by a black arrow, has no detectable mCherry expression. FIG. 7F) The dorsal view of a dissected abdomen, including liver and the large and small intestines, which do not express detectable levels of mCherry, compared with the mCherry expressing abdominal muscle in view at the left of the frame.

FIGS. 8A-8L depict the HLH cassette displaying extremely high activity in most fast-twitch muscles, moderate activity in EDL, soleus, and diaphragm, and low activity in heart. The mCherry reporter under control of HLH was delivered systemically by retro-orbital injection to wild-type mice using rAAVrh74 virions at 2.6×10¹⁴ GC/kg. The fluorescent signal was visualized at 12 weeks post-injection with an exposure time of 1 second (1 s). rAAV-injected tissues are indicated by asterisks. For two-tissue panels FIGS. 8A-8B and 8D-8H, tissues from uninjected mice are shown on the left. For four-tissue panel FIG. 8C, uninjected tissues for soleus and EDL are on the left of each sub-panel. Single-tissue panels FIGS. 8J-8L show rAAV-injected; FIG. 8I shows uninjected. Abbreviations: tibialis anterior (TA), gastrocnemius (GA), quadriceps (QUA), soleus (SOL), extensor digitorum longus (EDL).

FIGS. 9A-9J depict the HLH cassette having no detectable activity in non-muscle tissues. Non-muscle tissues from the rAAVrh74-injected wild-type mice assayed in FIG. 8 were similarly assayed for mCherry expression and imaged at 0.5-second exposure. FIGS. 9A-9J show tissues from rAAV-injected mice. FIG. 9A. Hindlimb skeletal muscles, including the posterior biceps femoris, quadriceps, and gastrocnemius, express mCherry, while the sciatic nerve, indicated by a black arrow, has no detectable mCherry expression. FIG. 9F. The dorsal view of a dissected abdomen, including liver and the large and small intestines, which do not express detectable levels of mCherry, compared with the mCherry expressing abdominal muscle in view at the left of the frame.

### DETAILED DESCRIPTION

### Definitions

Unless defined otherwise, all technical and scientific terms used herein have the same meaning as commonly understood by one of ordinary skill in the art to which the invention pertains. Although any methods and materials similar or equivalent to those described herein can be used in the practice for testing of the present invention, the preferred materials and methods are described herein. In describing and claiming the present invention, the following terminology will be used.

It is also to be understood that the terminology used herein is for the purpose of describing particular embodiments only, and is not intended to be limiting.

The articles “a” and “an” are used herein to refer to one or to more than one (i.e., to at least one) of the grammatical object of the article. By way of example, “an element” means one element or more than one element.

“About” as used herein when referring to a measurable value such as an amount, a temporal duration, and the like, is meant to encompass variations of ±20% or ±10%, more preferably ±5%, even more preferably ±1%, and still more preferably ±0.1% from the specified value, as such variations are appropriate to perform the disclosed methods.

The term “AAV vector” as used herein refers to a polynucleotide vector comprising one or more genes of interest (or transgenes) that are flanked by AAV terminal repeat sequences (ITRs). AAV vectors can be produced and packaged into infectious viral particles when present in a host cell that has been transfected with one or more helper plasmids encoding and expressing rep and cap proteins and one or more proteins from adenovirus open reading frame E4orf6. The AAV vectors may be operably linked to promoter and enhancer sequences that can regulate the expression of the protein encoded by the AAV vector.

The terms “AAV virion” or “AAV viral particle” or “AAV vector particle” as used herein refers to a viral particle composed of capsid proteins from at least one AAV serotype surrounding a polynucleotide AAV vector. If the particle comprises a heterologous polynucleotide (i.e. a polynucleotide other than a wild-type AAV genome such as a transgene to be delivered to a mammalian cell), it is typically referred to as an “AAV vector particle” or simply an “AAV vector”. Thus, production of AAV vector particle necessarily includes production of AAV vector, as such a vector is contained within an AAV vector particle.

The term “Packaging” as used herein refers to intracellular process by which viral virions or particles (e.g. AAV virions or particles), especially viral vector particles or virions are assembled in a host cell. “Packaging” cells comprise the polynucleotide (e.g. helper plasmids) and protein components necessary to assemble functional viral virions.

By “agent” is meant any nucleic acid molecule, small molecule chemical compound, antibody, or polypeptide, or fragments thereof.

By “alteration” or “change” is meant an increase or decrease. An alteration may be by as little as 1%, 2%, 3%, 4%, 5%, 10%, 20%, 30%, or by 40%, 50%, 60%, or even by as much as 70%, 75%, 80%, 90%, or 100%.

As used herein, the term “autologous” is meant to refer to any material derived from the same individual to which it is later to be re-introduced into the individual.

“Allogeneic” refers to a graft derived from a different animal of the same species.

“Xenogeneic” refers to a graft derived from an animal of a different species.

By “biologic sample” is meant any tissue, cell, fluid, or other material derived from an organism.

As used herein, the term “cassette” or “expression cassette” or “regulatory cassette” refer to distinct nucleic acid vectors consisting of a payload sequence (e.g. encoding a transgene or RNA) and regulatory sequences (i.e. promoter, enhancers, terminators, and the like) which control its expression. Upon successful insertion into a host cell, the regulatory sequences allow for the transcription and translation of the payload transgene.

As used herein, the term “conservative sequence modifications” is intended to refer to amino acid modifications that do not significantly affect or alter the binding characteristics of the antibody containing the amino acid sequence. Such conservative modifications include amino acid substitutions, additions and deletions. Modifications can be introduced into an antibody of the invention by standard techniques known in the art, such as site-directed mutagenesis and PCR-mediated mutagenesis. Conservative amino acid substitutions are ones in which the amino acid residue is replaced with an amino acid residue having a similar side chain. Families of amino acid residues having similar side chains have been defined in the art. These families include amino acids with basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine, tryptophan), nonpolar side chains (e.g., alanine, valine, leucine, isoleucine, proline, phenylalanine, methionine), beta-branched side chains (e.g., threonine, valine, isoleucine) and aromatic side chains (e.g., tyrosine, phenylalanine, tryptophan, histidine). Thus, one or more amino acid residues within the CDR regions of an antibody can be replaced with other amino acid residues from the same side chain family and the altered antibody can be tested for the ability to bind antigens using the functional assays described herein.

A “disease” is a state of health of an animal wherein the animal cannot maintain homeostasis, and wherein if the disease is not ameliorated then the animal's health continues to deteriorate. In contrast, a “disorder” in an animal is a state of health in which the animal is able to maintain homeostasis, but in which the animal's state of health is less favorable than it would be in the absence of the disorder. Left untreated, a disorder does not necessarily cause a further decrease in the animal's state of health.

“Effective amount” or “therapeutically effective amount” are used interchangeably herein, and refer to an amount of a compound, formulation, material, or composition, as described herein effective to achieve a particular biological result or provides a therapeutic or prophylactic benefit. Such results may include, but are not limited to, anti-tumor activity as determined by any means suitable in the art.

“Encoding” refers to the inherent property of specific sequences of nucleotides in a polynucleotide, such as a gene, a cDNA, or an mRNA, to serve as templates for synthesis of other polymers and macromolecules in biological processes having either a defined sequence of nucleotides (i.e., rRNA, tRNA and mRNA) or a defined sequence of amino acids and the biological properties resulting therefrom. Thus, a gene encodes a protein if transcription and translation of mRNA corresponding to that gene produces the protein in a cell or other biological system. Both the coding strand, the nucleotide sequence of which is identical to the mRNA sequence and is usually provided in sequence listings, and the non-coding strand, used as the template for transcription of a gene or cDNA, can be referred to as encoding the protein or other product of that gene or cDNA.

As used herein “endogenous” refers to any material from or produced inside an organism, cell, tissue or system.

As used herein, the term “exogenous” refers to any material introduced from or produced outside an organism, cell, tissue or system.

The term “expression” as used herein is defined as the transcription and/or translation of a particular nucleotide sequence driven by its promoter.

“Expression vector” refers to a vector comprising a recombinant polynucleotide comprising expression control sequences operatively linked to a nucleotide sequence to be expressed. An expression vector comprises sufficient cis-acting elements for expression; other elements for expression can be supplied by the host cell or in an in vitro expression system. Expression vectors include all those known in the art, such as cosmids, plasmids (e.g., naked or contained in liposomes) and viruses (e.g., Sendai viruses, lentiviruses, retroviruses, adenoviruses, and adeno-associated viruses) that incorporate the recombinant polynucleotide.

“Homologous” as used herein, refers to the subunit sequence identity between two polymeric molecules, e.g., between two nucleic acid molecules, such as, two DNA molecules or two RNA molecules, or between two polypeptide molecules. When a subunit position in both of the two molecules is occupied by the same monomeric subunit; e.g., if a position in each of two DNA molecules is occupied by adenine, then they are homologous at that position. The homology between two sequences is a direct function of the number of matching or homologous positions; e.g., if half (e.g., five positions in a polymer ten subunits in length) of the positions in two sequences are homologous, the two sequences are 50% homologous; if 90% of the positions (e.g., 9 of 10), are matched or homologous, the two sequences are 90% homologous.

“Identity” as used herein refers to the subunit sequence identity between two polymeric molecules particularly between two amino acid molecules, such as, between two polypeptide molecules. When two amino acid sequences have the same residues at the same positions; e.g., if a position in each of two polypeptide molecules is occupied by an Arginine, then they are identical at that position. The identity or extent to which two amino acid sequences have the same residues at the same positions in an alignment is often expressed as a percentage. The identity between two amino acid sequences is a direct function of the number of matching or identical positions; e.g., if half (e.g., five positions in a polymer ten amino acids in length) of the positions in two sequences are identical, the two sequences are 50% identical; if 90% of the positions (e.g., 9 of 10), are matched or identical, the two amino acids sequences are 90% identical.

As used herein, an “instructional material” includes a publication, a recording, a diagram, or any other medium of expression which can be used to communicate the usefulness of the compositions and methods of the invention. The instructional material of the kit of the invention may, for example, be affixed to a container which contains the nucleic acid, peptide, and/or composition of the invention or be shipped together with a container which contains the nucleic acid, peptide, and/or composition. Alternatively, the instructional material may be shipped separately from the container with the intention that the instructional material and the compound be used cooperatively by the recipient.

“Isolated” means altered or removed from the natural state. For example, a nucleic acid or a peptide naturally present in a living animal is not “isolated,” but the same nucleic acid or peptide partially or completely separated from the coexisting materials of its natural state is “isolated.” An isolated nucleic acid or protein can exist in substantially purified form, or can exist in a non-native environment such as, for example, a host cell.

By the term “modified” as used herein, is meant a changed state or structure of a molecule or cell of the invention. Molecules may be modified in many ways, including chemically, structurally, and functionally. Cells may be modified through the introduction of nucleic acids.

By the term “modulating,” as used herein, is meant mediating a detectable increase or decrease in the level of a response in a subject compared with the level of a response in the subject in the absence of a treatment or compound, and/or compared with the level of a response in an otherwise identical but untreated subject. The term encompasses perturbing and/or affecting a native signal or response thereby mediating a beneficial therapeutic response in a subject, preferably, a human.

In the context of the present invention, the following abbreviations for the commonly occurring nucleic acid bases are used. “A” refers to adenosine, “C” refers to cytosine, “G” refers to guanosine, “T” refers to thymidine, and “U” refers to uridine.

Unless otherwise specified, a “nucleotide sequence encoding an amino acid sequence” includes all nucleotide sequences that are degenerate versions of each other and that encode the same amino acid sequence. The phrase nucleotide sequence that encodes a protein or an RNA may also include introns to the extent that the nucleotide sequence encoding the protein may in some version contain an intron(s).

The term “operably linked” refers to functional linkage between a regulatory sequence and a heterologous nucleic acid sequence resulting in expression of the latter. For example, a first nucleic acid sequence is operably linked with a second nucleic acid sequence when the first nucleic acid sequence is placed in a functional relationship with the second nucleic acid sequence. For instance, a promoter is operably linked to a coding sequence if the promoter affects the transcription or expression of the coding sequence. Generally, operably linked DNA sequences are contiguous and, where necessary to join two protein coding regions, in the same reading frame.

“Parenteral” administration of an immunogenic composition includes, e.g., subcutaneous (s.c.), intravenous (i.v.), intramuscular (i.m.), or intrasternal injection, or infusion techniques. The term “polynucleotide” as used herein is defined as a chain of nucleotides. Furthermore, nucleic acids are polymers of nucleotides. Thus, nucleic acids and polynucleotides as used herein are interchangeable. One skilled in the art has the general knowledge that nucleic acids are polynucleotides, which can be hydrolyzed into the monomeric “nucleotides.” The monomeric nucleotides can be hydrolyzed into nucleosides. As used herein polynucleotides include, but are not limited to, all nucleic acid sequences which are obtained by any means available in the art, including, without limitation, recombinant means, i.e., the cloning of nucleic acid sequences from a recombinant library or a cell genome, using ordinary cloning technology and PCR™, and the like, and by synthetic means.

As used herein, the terms “peptide,” “polypeptide,” and “protein” are used interchangeably, and refer to a compound comprised of amino acid residues covalently linked by peptide bonds. A protein or peptide must contain at least two amino acids, and no limitation is placed on the maximum number of amino acids that can comprise a protein's or peptide's sequence. Polypeptides include any peptide or protein comprising two or more amino acids joined to each other by peptide bonds. As used herein, the term refers to both short chains, which also commonly are referred to in the art as peptides, oligopeptides and oligomers, for example, and to longer chains, which generally are referred to in the art as proteins, of which there are many types. “Polypeptides” include, for example, biologically active fragments, substantially homologous polypeptides, oligopeptides, homodimers, heterodimers, variants of polypeptides, modified polypeptides, derivatives, analogs, fusion proteins, among others. The polypeptides include natural peptides, recombinant peptides, synthetic peptides, or a combination thereof.

The term “promoter” as used herein is defined as a DNA sequence recognized by the synthetic machinery of the cell, or introduced synthetic machinery, required to initiate the specific transcription of a polynucleotide sequence.

As used herein, the term “promoter/regulatory sequence” means a nucleic acid sequence which is required for expression of a gene product operably linked to the promoter/regulatory sequence. In some instances, this sequence may be the core promoter sequence and in other instances, this sequence may also include an enhancer sequence and other regulatory elements which are required for expression of the gene product. The promoter/regulatory sequence may, for example, be one which expresses the gene product in a tissue specific manner.

A “constitutive” promoter is a nucleotide sequence which, when operably linked with a polynucleotide which encodes or specifies a gene product, causes the gene product to be produced in a cell under most or all physiological conditions of the cell.

An “inducible” promoter is a nucleotide sequence which, when operably linked with a polynucleotide which encodes or specifies a gene product, causes the gene product to be produced in a cell substantially only when an inducer which corresponds to the promoter is present in the cell.

A “tissue-specific” promoter is a nucleotide sequence which, when operably linked with a polynucleotide encodes or specified by a gene, causes the gene product to be produced in a cell substantially only if the cell is a cell of the tissue type corresponding to the promoter.

The term “epigenetic” as used herein refers to heritable influences on gene expression that do not involve alterations in DNA nucleotide sequence. Epigenetic regulation can enhance or inhibit expression of affected genes, and can involve chemical modifications of the deoxyribose backbone of the DNA or the association of DNA/histone protein complexes or both.

The term “epigenetic regulator” as used herein refers to factors, enzymes, compounds, or compositions that act to alter the epigenetic status of a specific DNA locus. Epigenetic regulators can induce or catalyze the modification of DNA-associated proteins or the chemical structure of the DNA itself.

The terms “epigenetic tag” or “epigenetic marker” or “epigenetic mark” as used interchangeably herein, describe the specific chemical modifications made to DNA and DNA-associated proteins that result in epigenetic regulation of gene expression. Examples of epigenetic marks or tags can include but are not limited to the addition or removal of methyl or acetyl groups from CpG dinucleotides and histone proteins. The number and density of epigenetic tags or marks may correlate with the degree of epigenetic regulation a particular DNA locus is subject to.

A “signal transduction pathway” refers to the biochemical relationship between a variety of signal transduction molecules that play a role in the transmission of a signal from one portion of a cell to another portion of a cell. The phrase “cell surface receptor” includes molecules and complexes of molecules capable of receiving a signal and transmitting signal across the plasma membrane of a cell.

The term “tropism”, as used herein, refers to the cell types or tissue types that can be productively infected by a particular virus or virus-based vector (e.g. an AAV vector). Though not necessarily exclusive, tropism is often expressed as a “preference” or “specificity” of a virus or viral vector for a particular cell or tissue type. Being parasitic entities, cellular or tissue tropism is one of the major characteristics of specific viruses, which often require insertion into particular cell types in order to maintain a successful infectious cycle in target cells. When referring to viral vectors (e.g. AAV vectors), tissue or cellular tropism or specificity or preference can be used to direct therapeutic payloads to specific tissue or cell types.

By the term “specifically binds,” as used herein with respect to an antibody, is meant an antibody which recognizes a specific antigen, but does not substantially recognize or bind other molecules in a sample. For example, an antibody that specifically binds to an antigen from one species may also bind to that antigen from one or more species. But such cross-species reactivity does not itself alter the classification of an antibody as specific. In another example, an antibody that specifically binds to an antigen may also bind to different allelic forms of the antigen. However, such cross reactivity does not itself alter the classification of an antibody as specific. In some instances, the terms “specific binding” or “specifically binding,” can be used in reference to the interaction of an antibody, a protein, or a peptide with a second chemical species, to mean that the interaction is dependent upon the presence of a particular structure (e.g., an antigenic determinant or epitope) on the chemical species; for example, an antibody recognizes and binds to a specific protein structure rather than to proteins generally. If an antibody is specific for epitope “A”, the presence of a molecule containing epitope A (or free, unlabeled A), in a reaction containing labeled “A” and the antibody, will reduce the amount of labeled A bound to the antibody.

The term “subject” is intended to include living organisms in which an immune response can be elicited (e.g., mammals). A “subject” or “patient,” as used therein, may be a human or non-human mammal. Non-human mammals include, for example, livestock and pets, such as ovine, bovine, porcine, canine, feline and murine mammals. Preferably, the subject is human.

A “target site” or “target sequence” refers to a genomic nucleic acid sequence that defines a portion of a nucleic acid to which a binding molecule may specifically bind under conditions sufficient for binding to occur.

The term “therapeutic” as used herein means a treatment and/or prophylaxis. A therapeutic effect is obtained by suppression, remission, or eradication of a disease state.

The term “transfected” or “transformed” or “transduced” as used herein refers to a process by which exogenous nucleic acid is transferred or introduced into the host cell. A “transfected” or “transformed” or “transduced” cell is one which has been transfected, transformed or transduced with exogenous nucleic acid. The cell includes the primary subject cell and its progeny.

The term “transgene” refers to the genetic material that has been or is about to be artificially inserted into the genome of an animal, particularly a mammal and more particularly a mammalian cell of a living animal.

The term “transgenic animal” refers to a non-human animal, usually a mammal, having a non-endogenous (i.e., heterologous) nucleic acid sequence present as an extrachromosomal element in a portion of its cells or stably integrated into its germ line DNA (i.e., in the genomic sequence of most or all of its cells), for example a transgenic mouse. A heterologous nucleic acid is introduced into the germ line of such transgenic animals by genetic manipulation of, for example, embryos or embryonic stem cells of the host animal.

The term “knockout mouse” refers to a mouse that has had an existing gene inactivated (i.e. “knocked out”). In some embodiments, the gene is inactivated by homologous recombination. In some embodiments, the gene is inactivated by replacement or disruption with an artificial nucleic acid sequence.

To “treat” a disease as the term is used herein, means to reduce the frequency or severity of at least one sign or symptom of a disease or disorder experienced by a subject.

The phrase “under transcriptional control” or “operatively linked” as used herein means that the promoter is in the correct location and orientation in relation to a polynucleotide to control the initiation of transcription by RNA polymerase and expression of the polynucleotide.

A “vector” is a composition of matter which comprises an isolated nucleic acid and which can be used to deliver the isolated nucleic acid to the interior of a cell. Numerous vectors are known in the art including, but not limited to, linear polynucleotides, polynucleotides associated with ionic or amphiphilic compounds, plasmids, and viruses. Thus, the term “vector” includes an autonomously replicating plasmid or a virus. The term should also be construed to include non-plasmid and non-viral compounds which facilitate transfer of nucleic acid into cells, such as, for example, polylysine compounds, liposomes, and the like. Examples of viral vectors include, but are not limited to, Sendai viral vectors, adenoviral vectors, adeno-associated virus vectors, retroviral vectors, lentiviral vectors, and the like.

Ranges: throughout this disclosure, various aspects of the invention can be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 2.7, 3, 4, 5, 5.3, and 6. This applies regardless of the breadth of the range.

### Description

The present invention is based on the unexpected observation that muscle-specific regulatory cassettes can be constructed which significantly and preferentially express payload genes comprised within the cassettes in certain muscle tissues. In certain embodiments, the muscle-specific regulatory cassette of the invention efficiently expresses the payload gene in skeletal muscle tissue but not in cardiac tissue. Also included in the invention are muscle-specific regulatory cassettes which also have robust payload gene expression in skeletal muscle tissue with lower but still detectable levels of expression in cardiac tissue.

The regulatory cassettes of the present invention have also been minimized with respect to size, such that they are smaller than other muscle-specific regulatory cassettes known in the art. In this way, these cassettes maximize the size of the payload gene which can be packaged into cell-transduction systems, such as AAV vector systems and the like. As such, in certain embodiments, the regulatory cassettes of the invention can be comprised within AAV vector systems, which efficiently transfer the regulatory cassette and its associated transgene into mammalian cells and tissues.

### Cardiac Toxicity is a Challenge to Successful Gene Therapies

Gene therapy approaches in preclinical models of muscle disease can result in cardiotoxicity caused by cardiac expression of the transgene. While this toxicity can be prevented with the use of muscle-specific regulatory cassettes in conjunction with miRNA detargeting sequences, together these elements add considerable size to therapeutic cassettes already limited by the stringent packaging capacity of recombinant AAV (rAAV) vectors. Of even greater concern are the severe adverse events reported in recent gene therapy trials for two muscle diseases. Deaths in the ASPIRO X-linked myotubular myopathy (XLMTM) clinical trial were due to liver failure, but several patients had laboratory findings indicative of myocarditis, and in a Pfizer trial for Duchenne Muscular Dystrophy (DMD), two instances of myocarditis were reported. Thus, there is a clinical need for minimized gene regulatory cassettes with high activity in skeletal muscle, but low or no activity in heart.

Previous studies have found that the regulatory regions of the muscle creatine kinase (Ckm) gene, which mediate transcription, are useful for directing highly tissue-specific expression in skeletal and cardiac muscle tissues (Salva et al. (2007) Mol Ther, 2007. 15 (2): p. 320-9.). Regulatory cassettes comprising sequences derived from these regions have been demonstrated to drive robust, tissue-specific transgene expression.

In certain embodiments, the present invention includes Muscle creatine kinase (CKM)-based regulatory cassettes which have undergone multiple modifications in order to alter their tissue specificities beyond currently-available cassette systems: 1) No Heart (NH), a cassette that is highly active in most fast-twitch skeletal muscles, but essentially inactive in soleus (slow-twitch muscle), diaphragm, and heart; and 2) Have a Little Heart (HLH), a cassette that displays extremely high activity in fast-twitch muscles, with moderate activity in soleus and diaphragm, and low activity in heart. Thus, the NH cassette will be useful for skeletal muscle indications where no cardiac expression is needed or can be tolerated, whereas the HLH cassette will be useful for providing transgene expression in all striated muscles while avoiding cardiotoxicity associated with high transgene expression. Together, these cassettes are applicable to diverse gene therapy strategies targeting a broad range of myopathies.

### AAV Vectors

AAV are relatively small, non-enveloped viruses with a ˜4 kb genome that is flanked by inverted terminal repeats (ITRs). The genome contains two open reading frames, one of which provides proteins necessary for replication and the other provides components required for construction of the viral capsid. Wild-type AAV is typically found in the presence of adenovirus as the adenoviruses provide helper proteins that are essential for packaging of the AAV genome into virions. Therefore, AAV production piggy-backs on co-infection with adenovirus and relies on three key elements: the ITR-flanked genome, the open-reading frames, and adeno-helper genes. Due to their non-pathogenic ability to readily infect human cells, AAV is well-studied as a vector for gene delivery. AAV may be readily obtained and their use as vectors for gene delivery has been described in, for example, Muzyczka, 1992; U.S. Pat. No. 4,797,368, and PCT Publication WO 91/18088. Construction of AAV vectors is described in a number of publications, including Lebkowski et al., 1988; Tratschin et al., 1985; Hermonat and Muzyczka, 1984.

AAV-based vector systems typically separate the viral AAV genes, Adenovirus-derived helper genes, and the transgene payload onto two or three separate plasmids. Three plasmid systems consist of an AAV helper plasmid comprising the rep (replication) and cap (capsid) genes, an adenoviral helper plasmid comprising at least the E2a gene, E4 gene, and VA (viral associated) RNA, and a payload plasmid comprising the transgene and associated promoters and enhancers flanked by ITR sequences. The helper plasmid or plasmids do not comprise ITRs in order to prevent packaging of a functional, infectious viral genome.

Two plasmid systems combine the AAV rep and cap genes and adenoviral helper genes onto a single plasmid and simplify viral vector production by reducing the number of transfected plasmids. Often, a dedicated packaging cell line is used which is engineered to express AAV/helper genes prior to introduction of the payload plasmid.

Successful gene therapies require efficient infection of target tissues and establishment of long term gene expression, and previous studies have demonstrated that AAV vectors can successfully infect and transduce a broad variety of cell and tissue types, such as brain, liver, and muscle, among others, and have the ability to infect both dividing and quiescent cells. Additionally, AAV-mediated transduction of tissues has been demonstrated to result in long term transgene expression greater than 1.5 years in animal models including canine, murine and hamster.

The tissue tropism of AAV vector particles is influenced by the serotype of the capsid protein, though the receptors and co-receptors that the capsid proteins bind to are often poorly understood and can be expressed by multiple tissue types. For example, AAV2, one of the most well-studied serotypes, has a binding affinity largely for heparan sulfate proteoglycan (HSPG) and as such has a tropism in humans for eye, brain, lung, liver, muscle, and joint tissues. Likewise, AAVs 1, 4, 5, and 6 have a binding affinity largely for sialic acid and a tropism for neuronal tissues while AAVs 5 and 8 share a tropism for skeletal muscle. In this way, the serotype of the AAV capsid protein can be selected to target the payload nucleic acid (e.g. a regulatory cassette) of the AAV vector to a specific tissue or cell type. Alteration or modification of capsid protein structure can also alter the tissue or cellular tropism and affinity of the resulting AAV vector particles.

In certain preferred embodiments, the current invention comprises AAV vectors comprising a capsid protein derived from AAV9 and variants thereof. AAV9 and capsid proteins based on AAV9 transduce muscle, liver, and lung tissues about 100-fold more efficiently than AAV2, while also being able to cross the blood brain barrier.

In certain preferred embodiments, the current invention comprises AAV vectors comprising a capsid protein derived from AAVrh74 and variants thereof. AAVrh74 and capsid proteins based on AAVrh74 have a tropism for skeletal and cardiac tissue and are useful for delivering therapeutic nucleic acids to those tissues.

In certain preferred embodiments, the current invention comprises AAV vectors comprising a capsid protein derived from a MyoAAV capsid and variants thereof. MyoAAV are a family of capsid proteins which share a common arginine-glycine-aspartic acid (RGD) motif and were generated via directed evolution to select for high specificity and expression in muscle tissues, especially as compared to AAV9. Among the MyoAAV capsids that are known in the art include, but are not limited to MyoAAV 1A, the so-called second generation capsid MyoAAV 2 or 2A, MyoAAV 3A, MyoAAV 4A, MyoAAV 4C, and MyoAAV4E. Given the tissue specificities of MyoAAV family capsids for muscle tissues, it is contemplated that any MyoAAV capsid could be used with the therapeutic cassettes, vectors, compositions, and methods of the current invention. In certain preferred embodiments, the AAV vectors of the current invention comprise capsid proteins derived from MyoAAV2 or variants thereof.

It is contemplated that the regulatory cassettes of the invention can be used with any naturally occurring, modified, hybrid, or engineered AAV capsid protein that provides the desired tissue tropism including, but not limited to AAV1, AAV2, AAV3, AAV3B, AAV4, AAV5, AAV6, AAV7, AAV8, AAV9, AAV12, AAV-B1, AAV-DJ, AAV-Retro, AAVrh8, AAVrh10, AAVrh25, Anc80L65, LK03, AAVrh18, AAVrh74, AAVrh32.33, AAVrh39, AAVrh43, MyoAAV2, Oligo001, PHP-B, and Spark 100 among others or variants thereof. The skilled artisan would be able to select an appropriate capsid protein for use with the invention based on the desired target tissue or cell type.

### Nucleic Acids and Vectors

The present disclosure provides an isolated polynucleic acid encoding a muscle-specific regulatory cassette. In certain embodiments, the muscle-specific regulatory cassette comprises at least three modified enhancer sequences upstream and operably linked to a promoter, wherein the three modified enhancer sequences comprise a Trex sequence, an AT-rich sequence, a right E-box sequence, and a MEF2 sequence, wherein the right E-box sequence is repeated at least once, and wherein the promoter is a −80 to +50 promoter containing a consensus Inr sequence.

In certain embodiments, the muscle-specific regulatory cassette is encoded by a nucleic acid comprising a polynucleotide sequence having at least 80%, 85%, 90%, 95%, 96%, 96%, 97%, 98%, 99% identity to SEQ ID NO: 1.

In certain embodiments, the muscle-specific regulatory cassette is encoded by a nucleic acid comprising the polynucleotide sequence set forth in SEQ ID NO: 1.

In certain embodiments, the muscle-specific regulatory cassette is encoded by a nucleic acid consisting of the polynucleotide sequence set forth in SEQ ID NO: 1.

In certain embodiments, the muscle-specific regulatory cassette comprises at least three modified enhancer sequences upstream and operably linked to a promoter, wherein the at least three modified enhancer sequences comprise a Trex sequence, an AT-rich sequence, a left E-box sequence, a right E-box sequence, and a MEF2 sequence, and wherein the promoter is a −80 to +50 promoter containing a consensus Inr sequence.

In certain embodiments, the muscle-specific regulatory cassette is encoded by a nucleic acid comprising a polynucleotide sequence having at least 80%, 85%, 90%, 95%, 96%, 96%, 97%, 98%, 99% identity to SEQ ID NO: 2.

In certain embodiments, the muscle-specific regulatory cassette is encoded by a nucleic acid comprising the polynucleotide sequence set forth in SEQ ID NO: 2.

In certain embodiments, the muscle-specific regulatory cassette is encoded by a nucleic acid consisting of the polynucleotide sequence set forth in SEQ ID NO: 2.

### Gene Transfer Systems and Adeno-Associated Virus (AAV)

Gene transfer systems, such as those described in the present invention, depend upon a vector or vector system to shuttle the genetic constructs into target cells. Methods of introducing a nucleic acid into the hematopoietic stem or progenitor cell include physical, biological and chemical methods. Physical methods for introducing a polynucleotide, such as RNA, into a host cell include calcium phosphate precipitation, lipofection, particle bombardment, microinjection, electroporation, and the like. RNA can be introduced into target cells using commercially available methods which include electroporation (Amaxa Nucleofector-II (Amaxa Biosystems, Cologne, Germany)), (ECM 830 (BTX) (Harvard Instruments, Boston, Mass.) or the Gene Pulser II (BioRad, Denver, Colo.), Multiporator (Eppendort, Hamburg Germany). RNA can also be introduced into cells using cationic liposome mediated transfection using lipofection, using polymer encapsulation, using peptide mediated transfection, or using biolistic particle delivery systems such as “gene guns” (see, for example, Nishikawa, et al. Hum Gene Ther., 12 (8): 861-70 (2001).

Chemical means for introducing a polynucleotide into a host cell include colloidal dispersion systems, such as macromolecule complexes, nanocapsules, microspheres, beads, and lipid-based systems including oil-in-water emulsions, micelles, mixed micelles, and liposomes. An exemplary colloidal system for use as a delivery vehicle in vitro and in vivo is a liposome (e.g., an artificial membrane vesicle).

Lipids suitable for use can be obtained from commercial sources. For example, dimyristyl phosphatidylcholine (“DMPC”) can be obtained from Sigma, St. Louis, MO; dicetyl phosphate (“DCP”) can be obtained from K & K Laboratories (Plainview, NY); cholesterol (“Choi”) can be obtained from Calbiochem-Behring; dimyristyl phosphatidylglycerol (“DMPG”) and other lipids may be obtained from Avanti Polar Lipids, Inc. (Birmingham, AL). Stock solutions of lipids in chloroform or chloroform/methanol can be stored at about −20° C. Chloroform is used as the only solvent since it is more readily evaporated than methanol. “Liposome” is a generic term encompassing a variety of single and multilamellar lipid vehicles formed by the generation of enclosed lipid bilayers or aggregates. Liposomes can be characterized as having vesicular structures with a phospholipid bilayer membrane and an inner aqueous medium. Multilamellar liposomes have multiple lipid layers separated by aqueous medium. They form spontaneously when phospholipids are suspended in an excess of aqueous solution. The lipid components undergo self-rearrangement before the formation of closed structures and entrap water and dissolved solutes between the lipid bilayers (Ghosh et al., (1991) Glycobiology 5:505-10). However, compositions that have different structures in solution than the normal vesicular structure are also encompassed. For example, the lipids may assume a micellar structure or merely exist as nonuniform aggregates of lipid molecules. Also contemplated are lipofectamine-nucleic acid complexes.

Biological methods for introducing a polynucleotide of interest into a host cell include the use of DNA and RNA vectors. Viral vectors, and especially retroviral vectors, have become the most widely used method for inserting genes into mammalian, e.g., human cells. Other viral vectors can be derived from lentivirus, poxviruses, herpes simplex virus I, adenoviruses and adeno-associated viruses, and the like. See, for example, U.S. Pat. Nos. 5,350,674 and 5,585,362. Currently, the most efficient and effective way to accomplish the transfer of genetic constructs into living cells is through the use of vector systems based on viruses that have been made replication-defective. Some of the most effective vectors known in the art are those based on adeno-associated viruses (AAVs). AAVs are small viruses of the parvoviridae family that make attractive vectors for gene transfer in that they are replication defective, not known to cause any human disease, cause only a very mild immune response, can infect both actively dividing and quiescent cells, and stably persist in an extrachromosomal state without integrating into the target cell's genome. In certain embodiments, the present disclosure provides an AAV vector comprising the dCas9-based CRISPRi system of the invention.

Regardless of the method used to introduce the nucleic acid into the cell, a variety of assays may be performed to confirm the presence of the nucleic acid in the cell. Such assays include, for example, “molecular biological” assays well known to those of skill in the art, such as Southern and Northern blotting, RT-PCR and PCR; “biochemical” assays, such as detecting the presence or absence of a particular peptide, e.g., by immunological means (ELISAs and Western blots) or by assays described herein to identify agents falling within the scope of the invention.

### Methods of Treatment and Use

In certain embodiments, the present invention includes a method of treating a genetic disease or disorder in a subject in need thereof, comprising administering to the subject an effective amount of an AAV vector comprising a muscle-specific regulatory cassette comprising at least three modified enhancer sequences upstream and operably linked to a promoter, thereby treating the genetic disease or disorder, wherein the three modified enhancer sequences comprise a Trex sequence, an AT-rich sequence, a right E-box sequence, and a MEF2 sequence, wherein the right E-box sequence is repeated at least once, and wherein the promoter is a −80 to +50 promoter containing a consensus Inr sequence. As such, in certain embodiments, the AAV vector has a specificity or tropism for muscle tissue. In certain embodiments, the regulatory cassette is highly active in fast-twitch skeletal muscles with relatively low expression activity in the soleus and diaphragm muscles and no expression activity in cardiac tissues. As such, methods comprising these regulatory cassettes are ideally suited to the treatment of genetic diseases or disorders such as skeletal myopathies where cardiac muscle is unaffected (e.g., FSHD, XLMTM, central core myopathy, inclusion body myositis, most cases of nemaline myopathy, distal myopathy, centronuclear myopathy, and oculopharyngeal muscular dystrophy, and certain subtypes of LGMDs where patients show no cardiac involvement).

In certain embodiments, the invention of the present disclosure includes a method of treating a genetic disease or disorder in a subject in need thereof, comprising administering to the subject an effective amount of an AAV vector comprising a muscle-specific regulatory cassette comprising a polynucleotide encoding a muscle-specific regulatory cassette comprising at least three modified enhancer sequences upstream and operably linked to a promoter, wherein the at least three modified enhancer sequences comprise a Trex sequence, an AT-rich sequence, a left E-box sequence, a right E-box sequence, and a MEF2 sequence, wherein the promoter is a −80 to +50 promoter containing a consensus Inr sequence. As such, in certain embodiments, the AAV vector has a specificity or tropism for muscle tissue. In certain embodiments, the regulatory cassette exhibits moderate to high activity in all skeletal muscles and low expression activity in cardiac tissue. In this way, these cassettes provide moderate to high-level expression across all skeletal muscles in conjunction with low-level expression in heart, making them well-suited to the treatment of genetic diseases or disorders that affect all striated muscles, but where high expression of the therapeutic transgene is toxic in cardiac muscle. Examples of these include, but are not limited to DMD, XLMTM, and many subtypes of LGMD. For these diseases or disorders, effective treatment requires some therapeutic transgene expression in cardiac muscle, but low expression levels should prevent myocarditis.

In certain embodiments, the genetic disease or disorder which is treated by way of the methods of the current invention is a muscle-related genetic disease or disorder. Non-limiting examples of muscle-related genetic diseases or disorders which could be treated by the methods of the invention include, but are not limited to facioscapulohumeral muscular dystrophy (FSHD), X-linked myotubular myopathy (XLMTM), central core myopathy, inclusion body myositis, nemaline myopathy, distal myopathy, centronuclear myopathy, oculopharyngeal muscular dystrophy, a dysferlinopathy, a limb-girdle muscular dystrophy (LGMD), and Duchenne muscular dystrophy (DMD).

In certain embodiments, the invention includes a method of introducing a transgene into a target cell, comprising contacting the cell with a polynucleotide encoding a muscle-specific regulatory cassette comprising at least three modified enhancer sequences upstream and operably linked to a promoter, wherein the three modified enhancer sequences comprise a Trex sequence, an AT-rich sequence, a right E-box sequence, and a MEF2 sequence, wherein the right E-box sequence is repeated at least once, and wherein the promoter is a −80 to +50 promoter containing a consensus Inr sequence. In certain embodiments, the target cell is a mammalian muscle cell. In certain embodiments, the cassette has high expression activity in skeletal muscle cells. In certain preferred embodiments, the skeletal muscle tissue is fast-twitch muscle tissue. As used herein, the term “fast-twitch muscle tissue” refers to type II muscle tissues which are specialized for short, powerful contractions, as opposed to “slow-twitch” or type I muscle tissues which can sustain contractions for an extended period of time. In certain embodiments, the cassette has very low expression in cardiac tissue as compared to skeletal muscle tissue.

In certain embodiments, the invention includes a method of introducing a transgene into a target cell, comprising contacting the cell with a polynucleotide encoding a muscle-specific regulatory cassette comprising at least three modified enhancer sequences upstream and operably linked to a promoter, wherein the at least three modified enhancer sequences comprise a Trex sequence, an AT-rich sequence, a left E-box sequence, a right E-box sequence, and a MEF2 sequence, wherein the promoter is a −80 to +50 promoter containing a consensus Inr sequence. In certain embodiments, the target cell is a mammalian muscle cell. In certain embodiments, the cassette has high expression activity in skeletal muscle tissue accompanied by low-level, but still present, expression in cardiac tissues.

### Pharmaceutical Compositions

Pharmaceutical compositions of the present invention may comprise as described herein, in combination with one or more pharmaceutically or physiologically acceptable carriers, diluents, adjuvants or excipients. Such compositions may comprise buffers such as neutral buffered saline, phosphate buffered saline and the like; carbohydrates such as glucose, mannose, sucrose or dextrans, mannitol; proteins; polypeptides or amino acids such as glycine; antioxidants; chelating agents such as EDTA or glutathione; adjuvants (e.g., aluminum hydroxide); and preservatives. Compositions of the present invention are preferably formulated for intravenous administration.

Pharmaceutical compositions of the present disclosure may comprise the AAV vector particles as described herein, in combination with one or more pharmaceutically or physiologically acceptable carriers, diluents or excipients. Such compositions may comprise buffers such as neutral buffered saline, phosphate-buffered saline (PBS) and the like; carbohydrates such as glucose, mannose, sucrose or dextran, mannitol; proteins; polypeptides or amino acids such as glycine; antioxidants; chelating agents such as EDTA or glutathione; adjuvants (e.g., aluminum hydroxide); and preservatives. Compositions of the present disclosure are preferably formulated for a number of administration routes including oral, inhalation, nasal, nebulization, intravenous injection, intramuscular injection, intrathecal injection, intrapleural injection, intracisternal magna injection, subcutaneous injection, and/or transdermal injection. Pharmaceutical compositions of the present disclosure may be administered in a manner appropriate to the disease to be treated (or prevented). The quantity and frequency of administration will be determined by such factors as the condition of the patient, the type and severity of the patient's disease, and the type and functional nature of the patient's immune response to the phage particles, although appropriate dosages may be determined by clinical trials.

The AAV vector particles of the disclosure can be administered in dosages and routes and at times to be determined in appropriate pre-clinical and clinical experimentation and trials. Administration of the AAV vector particles of the disclosure may be combined with other methods useful to treat the desired disease or condition as determined by those of skill in the art.

In certain embodiments, the effective dose range is measured in units known to a person of skill in the art to be suitable for the description of AAV vector particle doses. In some embodiments, the effective dose range for a vaccine or therapeutic compound of the disclosure is measured by transducing units (TU)/kg/dose or genome copies (GC)/kg/dose or particles/kg/dose. In some embodiments, the dosage provided to a patient is between about 10⁶-10¹⁴ TU/kg. In some embodiments, the dosage provided to a patient is between about 10⁶-10¹⁴ GC/kg. In some embodiments, the effective dose range is measured by colony forming units (CFU), 50% tissue culture infectious dose (TCID₅₀), and combinations thereof.

Actual dosage levels of the active ingredients in the pharmaceutical compositions of this disclosure can be varied so as to obtain an amount of the active ingredient that is effective to achieve the desired therapeutic response for a particular patient, composition, and mode of administration, without being toxic to the patient.

The therapeutically effective amount or dose of a compound of the present disclosure depends on the age, sex and weight of the patient, the current medical condition of the patient and the progression of a disease or disorder contemplated in the disclosure.

A medical doctor, e.g., physician or veterinarian, having ordinary skill in the art can readily determine and prescribe the effective amount of the pharmaceutical composition required. For example, the physician or veterinarian could start doses of the compounds of the disclosure employed in the pharmaceutical composition at levels lower than that required in order to achieve the desired therapeutic effect and gradually increase the dosage until the desired effect is achieved.

In certain embodiments, the compositions of the disclosure are administered to the patient in dosages that range from one to five times per day or more. In other embodiments, the compositions of the disclosure are administered to the patient in range of dosages that include, but are not limited to, once every day, every two, days, every three days to once a week, and once every two weeks. It is readily apparent to one skilled in the art that the frequency of administration of the various combination compositions of the disclosure varies from individual to individual depending on many factors including, but not limited to, age, disease or disorder to be treated, gender, overall health, and other factors. Thus, the disclosure should not be construed to be limited to any particular dosage regime and the precise dosage and composition to be administered to any patient is determined by the attending physical taking all other factors about the patient into account.

Dosage size can be adjusted according to the weight, age, and stage of the disease of the subject being treated. AAV vector particles may also be administered multiple times at these dosages. The AAV vector particles can be administered by using infusion techniques that are commonly known in the art of immunotherapy or vaccinology. The optimal dosage and treatment regime for a particular patient can readily be determined by one skilled in the art of medicine by monitoring the patient for signs of disease and adjusting the treatment accordingly.

The administration of the AAV vector particle compositions of the disclosure may be carried out in any convenient manner known to those of skill in the art. The AAV vector particles of the present disclosure may be administered to a subject by aerosol inhalation, injection, ingestion, transfusion, implantation or transplantation. The compositions described herein may be administered to a subject or patient trans-arterially, subcutaneously, intranasally, intradermally, intratumorally, intranodally, intramedullary, intramuscularly, intravenously, or intraperitoneally. In other instances, the AAV vector particles of the disclosure are injected directly into a site of inflammation in the subject, a local disease site in the subject, a LN, an organ, a tumor, and the like. It should be understood that the method and compositions that would be useful in the present disclosure are not limited to the particular formulations set forth in the examples.

In certain embodiments, the compositions of the disclosure are formulated using one or more pharmaceutically acceptable excipients or carriers. In certain embodiments, the pharmaceutical compositions of the disclosure comprise a therapeutically effective amount of a compound of the disclosure and a pharmaceutically acceptable carrier.

The carrier can be a solvent or dispersion medium containing, for example, saline, buffered saline, water, ethanol, polyol (for example, glycerol, propylene glycol, and liquid polyethylene glycol, and the like), suitable mixtures thereof, and vegetable oils. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thimerosal and the like. In many cases, it is advisable to include isotonic agents, for example, sugars, sodium chloride, or polyalcohols such as mannitol and sorbitol, in the composition.

Formulations can be employed in admixtures with conventional excipients, i.e., pharmaceutically acceptable organic or inorganic carrier substances suitable for any suitable mode of administration, known to the art. The pharmaceutical preparations can be sterilized and if desired mixed with auxiliary agents, e.g., lubricants, preservatives, stabilizers, wetting agents, emulsifiers, salts for influencing osmotic pressure buffers, coloring, flavoring and/or aromatic substances and the like. They can also be combined where desired with other active agents, e.g., analgesic agents.

The practice of the present disclosure employs, unless otherwise indicated, conventional techniques of molecular biology (including recombinant techniques), microbiology, cell biology, biochemistry, and immunology, which are well within the purview of the skilled artisan. Such techniques are explained fully in the literature, such as, “Molecular Cloning: A Laboratory Manual”, fourth edition (Sambrook, 2012); 30 “Oligonucleotide Synthesis” (Gait, 1984); “Culture of Animal Cells” (Freshney, 2010); “Methods in Enzymology” “Handbook of Experimental Immunology” (Weir, 1997); “Gene Transfer Vectors for Mammalian Cells” (Miller and Calos, 1987); “Short Protocols in Molecular Biology” (Ausubel, 2002); “Polymerase Chain Reaction: Principles, Applications and Troubleshooting”, (Babar, 2011); “Current Protocols in Immunology” (Coligan, 2002). These techniques are applicable to the production of the polynucleotides and AAV particles of the disclosure, and, as such, may be considered in making and practicing the disclosure.

It should be understood that the method and compositions that would be useful in the present disclosure are not limited to the particular formulations set forth in the examples. The following examples are put forth so as to provide those of ordinary skill in the art with a complete disclosure and description of how to make and use the cells, expansion and culture methods, and therapeutic methods of the disclosure, and are not intended to limit the scope of what the inventors regard as their disclosure.

### Experimental Examples

The invention is further described in detail by reference to the following experimental examples. These examples are provided for purposes of illustration only, and are not intended to be limiting unless otherwise specified. Thus, the invention should in no way be construed as being limited to the following examples, but rather, should be construed to encompass any and all variations which become evident as a result of the teaching provided herein.

Without further description, it is believed that one of ordinary skill in the art can, using the preceding description and the following illustrative examples, make and utilize the compounds of the present invention and practice the claimed methods. The following working examples, therefore, specifically point out the preferred embodiments of the present invention, and are not to be construed as limiting in any way the remainder of the disclosure.

The materials and methods are now described.

Plasmids. The NH cassette (Himeda et al. (2021) Mol Ther Methods Clin Dev, 2021. 20: p. 298-311) consists of three copies of a modified Ckm enhancer in tandem, upstream of a modified Ckm promoter. Main features are as follows: 1) Left E-box mutated to Right E-box (Nguyen et al., (2003) J Biol Chem. 278 (47): p. 46494-505, and Hauser et al. (2000) Mol Ther. 2 (1): p. 16-25); 2) enhancer CArG and AP2 sites removed; 3) 63 bp between Right E-box and MEF2 site removed (Salva et al., (2007) Mol Ther. 15 (2): p 320-9); 4) sequence between transcription factor binding motifs minimized; 5) −80 (Donoviel et al., (1996) Mol Cell Biol. 16 (4): p 1649-58) to +50 (Salva et al., (2007) Mol Ther. 15 (2): p 320-9) promoter sequence used; and 6) consensus Initiator element (Inr) added (Salva et al. (2007) Mol Ther. 15 (2): p. 320-9). The HLH cassette is based on the NH cassette, with the following modification: the additional Right E-box was replaced with the original Left E-box from the Ckm enhancer. For both cassettes, the mCherry reporter was inserted downstream of the transcription start site. Reporter cassettes were synthesized and fully sequenced by GENEWIZ, LLC (South Plainfield, NJ). For evaluation of cassette activities in vivo, each regulatory cassette driving the mCherry reporter was cloned between the AAV2 ITRs (using MluI and RsrII) of the pAAV-CA plasmid (Menegas et al., (2015) Elife 4: pe10032), a gift from Naoshige Uchida (Addgene plasmid #69616; RRID: Addgene_69616). Recombinant AAVrh74 particles were produced by Vector Biolabs (Malvern, PA). Vector sequences are disclosed in Table 1.

Animals. All animal experiments were approved by the Institutional Animal Care and Use Committee of the University of Nevada, Reno. Three and half week-old male wild-type mice (C57BL/6 J) were anesthetized with 3.5% isoflurane before retro-orbital injection (ROI) of rAAVrh74 (2.6×10¹⁴ GC/kg) containing either the NH or HLH cassette regulating expression of the mCherry reporter.

In vivo optical imaging. The fur of the ventral abdominal area was removed with depilatory cream before imaging. The fluorescent images of mCherry in live mice under anesthesia (2%-isoflurane) were captured weekly for 10 weeks after ROI using the IVIS Lumina III and Living Image software ver. 4.3.1 (PerkinElmer) with λex=580 nm/λem=620 nm filters, exposure time of 1 second, 12.5×12.5-cm FOV, f/stop 4. All images were processed in Living Image equally with an epi-fluorescence scale of min=7×10⁸ and max=3.2×10¹⁰, smoothing=5×5 and binning=2. The total flux (photons/second) and maximum radiance (photons/second/cm²/steradian) were measured using Living Image software.

In vitro optical imaging. At 12 weeks post-injection, all mice were euthanized, tissues were dissected, and mCherry signals were captured with the Leica THUNDER/DFC-7000T fluorescent imaging system and Leica LAS X software, using the same exposure time, unless indicated in the figure. Images were assembled with Adobe Photoshop 24.1.0, and exposures were adjusted equally. The fluorescent signals in selected tissues were further measured using IVIS Lumina III and Living Image software with filters λex=580 nm/λem=620 nm, exposure time 0.5 second, FOV 4×4-cm, f/stop 8. The total flux (photons/second) and maximum radiance (photons/second/cm²/steradian) were measured using Living Image software.

Quantification of viral transduction. After in vitro imaging, genomic DNA was isolated from each tissue for assessment of rAAV infection. Viral genomes were quantified by qPCR (100 ng genomic DNA) using primers to the bovine growth hormone polyadenylation signal (bGH PAS) present in the transgene construct sequence and normalized to the endogenous single copy Rosa26 locus. Each tissue had three biological replicates. Oligonucleotide primer sequences are as reported (Himeda et al., (2021) Mol Ther Methods Clin Dev. 20: p. 298-311, and Himeda et al., (2016) Mol Ther. 24 (3): p. 527-35).

TABLE 1
Gene expression regulatory cassettes

| SEQ ID | | | |
|---|---|---|---|
| NO: | Name: | Type: | DNA Sequence: |
| 1 | NH | DNA | ggacacccgagaCgccCggttataattaaccccaacacctgcCcccccccccaaca |
| | Cassette | | cctgcCcgaCctctaaaaataacTcCTcggacacccgagaCgccCggttataatta |
| | | | accccaacacctgcCcccccccccaacacctgcCegaCctctaaaaataacTcCTc |
| | | | ggacacccgagaCgccCggttataattaaccccaacacctgcCcccccccccaaca |
| | | | cctgcCcgaCctctaaaaataacccctccctggggacaAcccctccCAgcCaAtAG |
| | | | cacAGCCtaggTCccCctatataaGGccaCggCTGCTGGCCCTTCCTCATTCTCAG |
| | | | TGTCACCTCCAGGATACAGACAGCCCCCCTTCAGCCCAGCCC |
| 2 | HLH | DNA | ggacacccgagaCgccCggttataattaaccAGGACACGTGGCGAccccccccaac |
| | Cassette | | acctgcCcgaCctctaaaaataacTcCTggacacccgagaCgccCggttataatta |
| | | | accAGGACACGTGGCGAccccccccaacacctgcCcgaCctctaaaaataacTcCT |
| | | | ggacacccgagaCgccCggttataattaaccAGGACACGTGGCGAccccccccaac |
| | | | acctgcCcgaCctctaaaaataacccctccctggggacaAcccctccCAgcCaAtA |
| | | | GcacAGCCtaggTCccCctatataaGGccaCggCTGCTGGCCCTTCCTCATTCTCA |
| | | | GTGTCACCTCCAGGATACAGACAGCCCCCCTTCAGCCCAGCCC |

The experimental results are now described.

### Example 1: No Heart (NH): A Muscle-Specific Cassette with High Activity in Fast-Twitch Muscles and No or Low Expression in Cardiac Muscle

The goal of the studies of the present disclosure was to design a highly minimized skeletal muscle-specific regulatory cassette for use in a CRISPR inhibition approach to Facioscapulohumeral muscular dystrophy (FSHD). To accommodate all therapeutic CRISPR components within a single rAAV vector required minimizing current regulatory cassettes.

Building on previous work (Salva, M. Z., et al., Mol Ther, 2007. 15 (2): p. 320-9. and Himeda, C. L., et al., Methods Mol Biol, 2011. 709: p. 3-19) and taking into account that cardiac muscle is not involved in FSHD, a minimized skeletal muscle regulatory cassette was designed to drive the expression of larger therapeutic components. Several muscle-specific regulatory cassettes optimized for expression in striated muscles have been developed (PCT application no: WO2022216988A2) and are being actively used in both academic and clinical settings. However, inconsistent nomenclature among users is a serious issue, with researchers often referring to different cassettes by the same name (e.g., the “tMCK” cassette in Addgene plasmid #105556 uses three copies of the wild-type (′km enhancer, while “tMCK” in Addgene plasmid #149410 uses three copies of a modified (′km enhancer). Although the significance of these differences is unclear, they can certainly complicate comparisons of efficacy and specificity between studies.

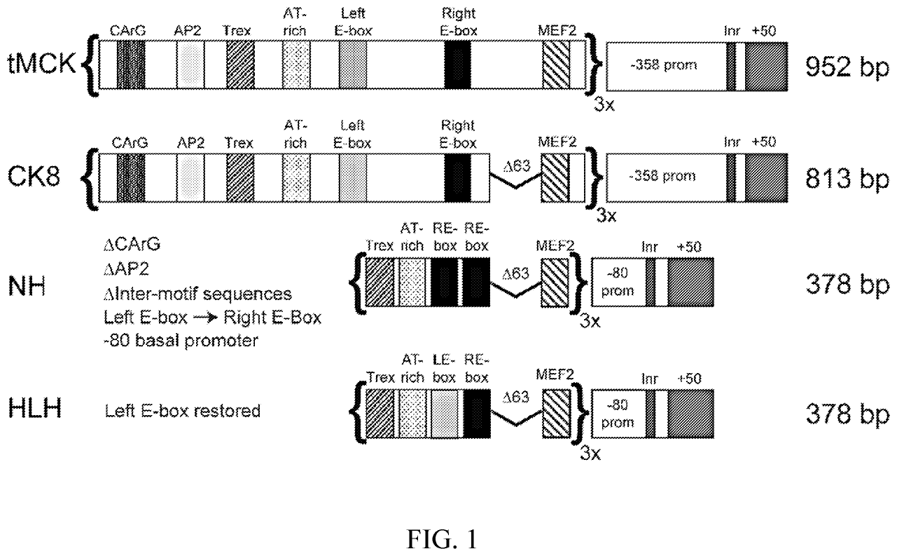

Starting with the well-established and widely used CK8 cassette, which is a modified version of three CKM enhancers upstream of the CKM promoter, additional sequences between elements were removed, and the CarG and AP2 sites, which are dispensable for expression in skeletal muscle, were deleted (FIG. 1). The Left E-box sequence was also mutated to create an additional Right E-box, a change that was reported to increase activity in skeletal myocytes (Nguyen et al., (2003) J Biol Chem. 278 (47): p 46494-505 and Hauser et al. (2000) Mol Ther. 2 (1): p 16-25), and the promoter was reduced to the 80-bp basal sequence (Donoviel et al., (1996) Mol Cell Biol. 16 (4): p 1649-58). This cassette was highly active in all skeletal muscles tested except for soleus, extensor digitorum longus (EDL), and diaphragm; it was virtually inactive in heart and in non-muscle tissues (Himeda et al. (2021) Mol Ther Methods Clin Dev. 20: p 298-311). Thus, we named this regulatory cassette No Heart (NH). Importantly, NH is active in most skeletal muscles, the critical target tissue for FSHD and a host of other skeletal myopathies, and the absence of heart expression is inconsequential for FSHD since heart is not pathologically affected (Galetta et al. (2005) Neuromuscul Disord. 15 (6): p 403-8; van Dijk et al. (2014) Funct Neurol. 29 (3): p 159-65; Ducharme-Smith et al. (2021) Front Neurol. 12: p 668180).

### Example 2: Have a Little Heart (HLH): A Muscle-Specific Cassette with Moderate to High Activity in all Anatomical Skeletal Muscles and Low Activity in Heart

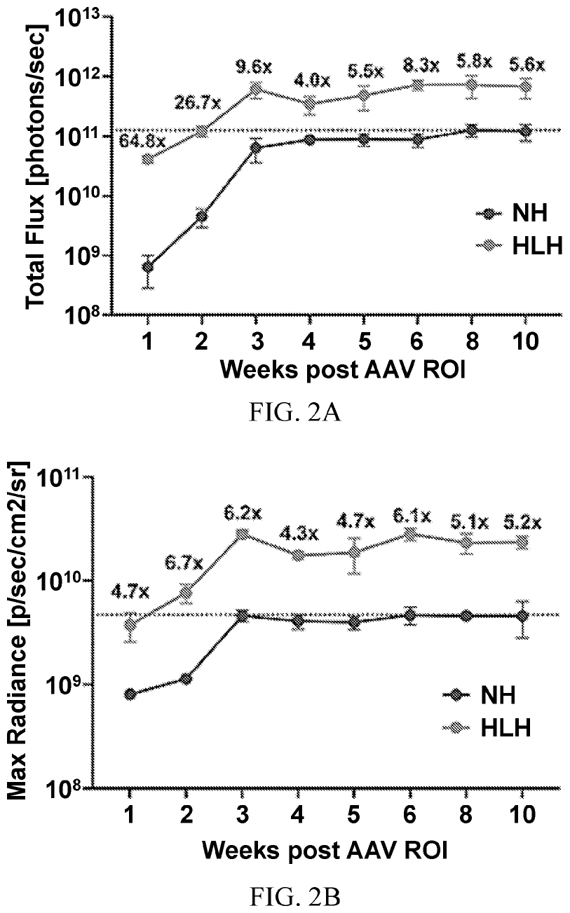

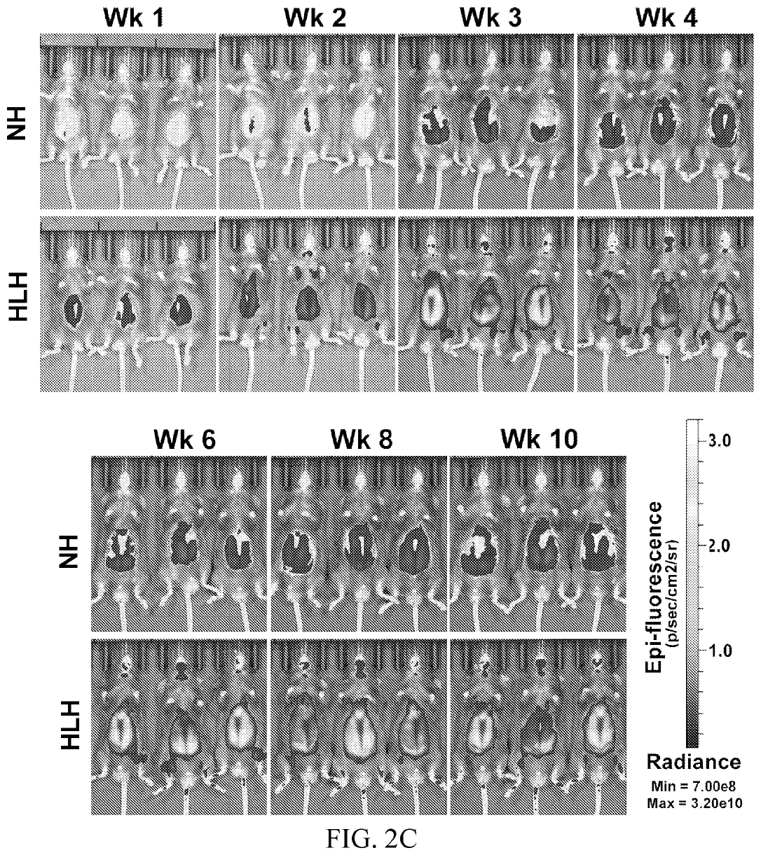

While NH is highly active in most fast-twitch muscles, one weakness is the extremely low activity in soleus, EDL, and diaphragm (Himeda et al. (2021) Mol Ther Methods Clin Dev. 20: p 298-311). To address this, we redesigned NH to replace the additional Right E-box with the original Left E-box from the (′km enhancer (Himeda, C. L., et al., Methods Mol Biol, 2011. 709: p. 3-19) (FIG. 1). Following the analysis described below, this cassette was named Have a Little Heart (HLH). In vivo activities of the two cassettes were compared directly using systemic rAAVrh74-mediated transgene delivery to wild-type mice. Viral particles were delivered by retro-orbital injection (2.6×10¹⁴ genome copy [GC]/kg body weight), and mCherry reporter activity (assessed as epi-fluorescence in abdominal and pectoral muscles) was monitored over time. Over ten weeks of monitoring, HLH showed much higher skeletal muscle activity than NH at every time point (FIG. 2). Most notably, HLH rapidly achieved high expression; within just two weeks of delivery, total flux of HLH (ave. 1.21×10¹¹) was already 95% of peak total flux of NH observed after 8 weeks of delivery (ave. 1.27×10¹¹), reaching 485% at 3 weeks (ave. 6.16×10¹¹) (FIG. 2A). The total flux is the epi-fluorescence signal (radiance) summed over the area where signal was detected, and may vary between animals; however, even by the maximum radiance measurement (the highest radiance for a pixel in the field), HLH outperformed NH (FIG. 2B); HLH showed 81% of peak NH maximum radiance by the first week (ave. 3.74×10⁹ vs 4.64×10⁹ of NH at 6 weeks), increased to 165% at 2 weeks (ave. 7.63×10⁹), and reached 606% at 3 weeks (ave. 2.81×10¹⁰) post ROI. Overall, HLH displayed 5-6× higher activity than NH in abdominal/pectoral muscles.

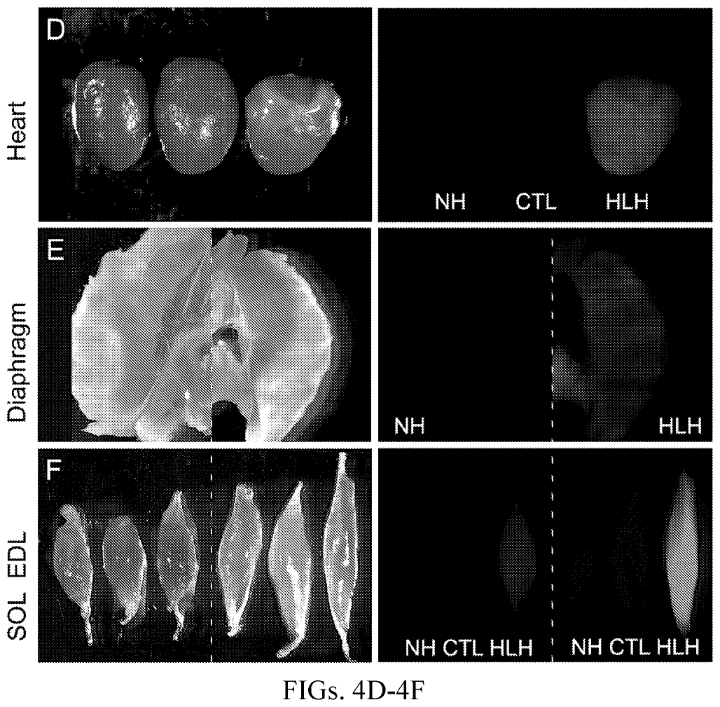

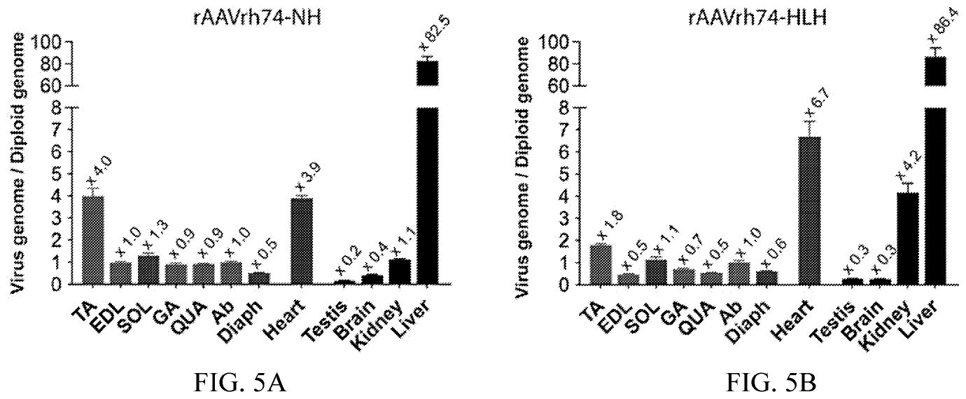

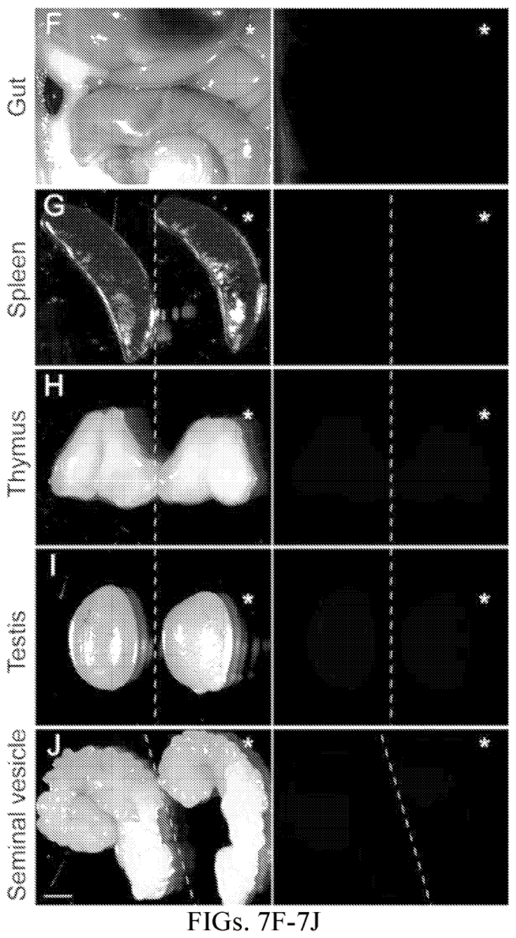

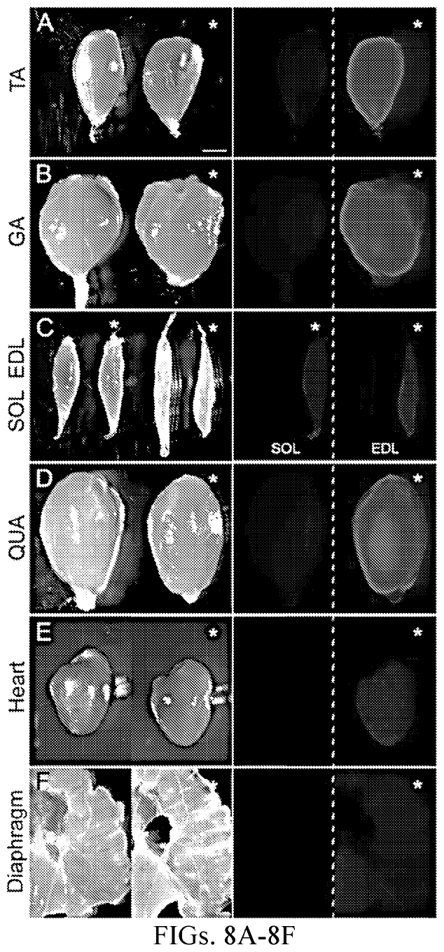

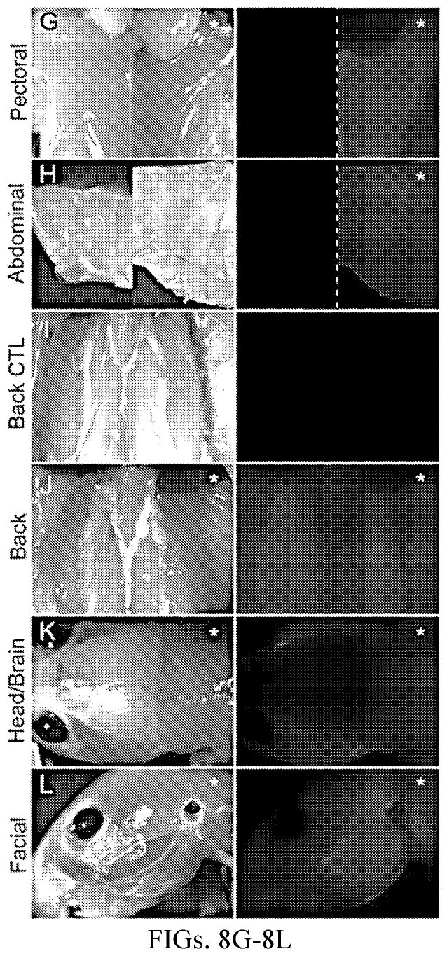

To further evaluate both cassettes in other muscles and tissues, mice were sacrificed at 12 weeks post-injection for in vitro epi-fluorescence quantification and fluorescence imaging. As previously reported (Pozsgai et al. (2017) Mol Ther. 25 (4): p 855-869; Potter et al. (2021) Hum Gene Ther. 32 (7-8): p 375-389), quantitative PCR (qPCR) for viral genomes present across tissues showed that rAAVrh74 strongly transduced liver, with moderate transduction of heart, kidney, and skeletal muscles (FIG. 5). Nonetheless, both cassettes maintained high activity across striated muscles, with HLH exhibiting 7.4-36.5× higher activity than NH, and no activity in non-muscle tissues (FIGS. 2-4, 6-9).

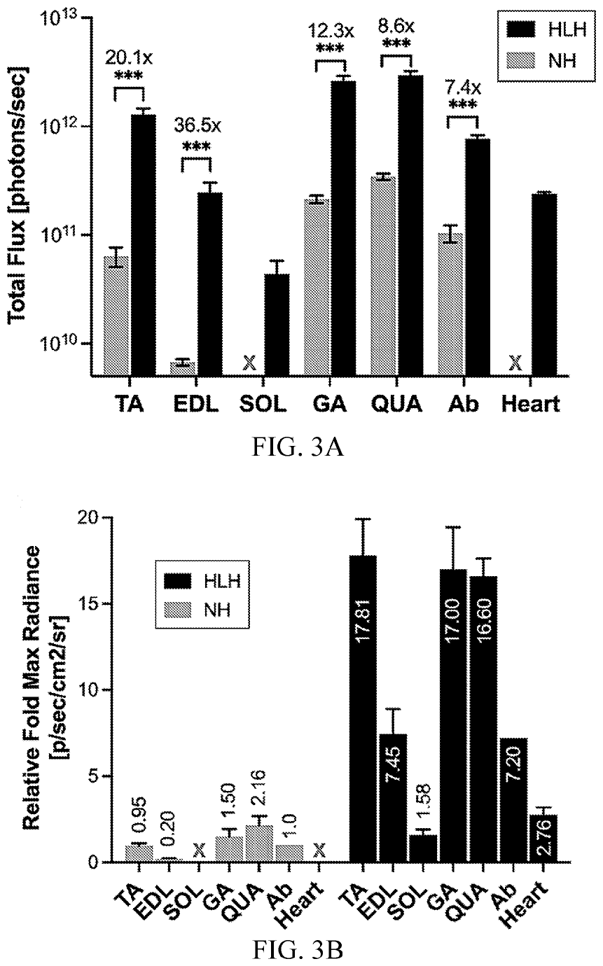

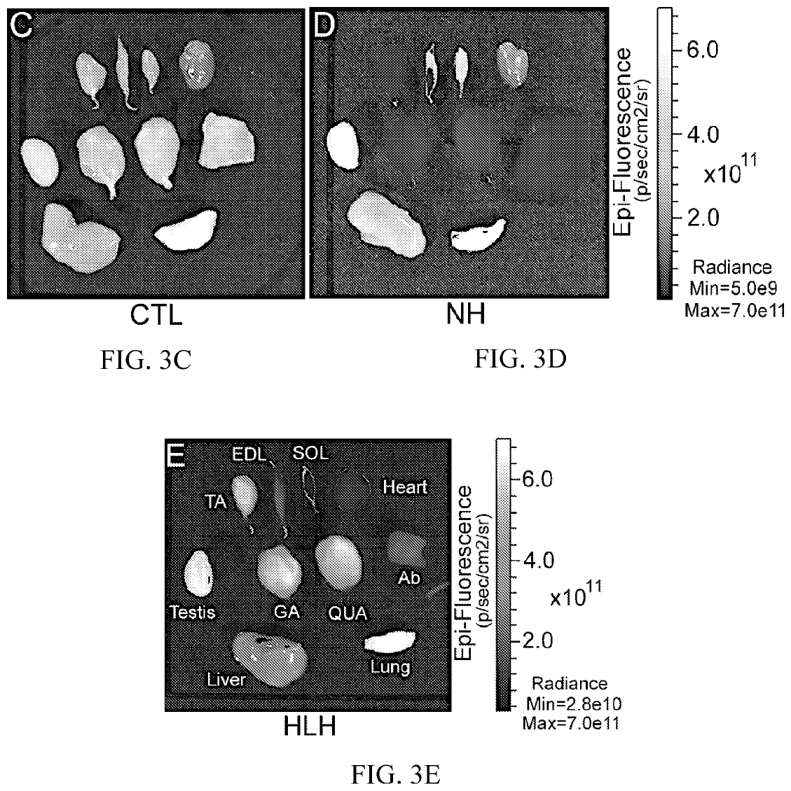

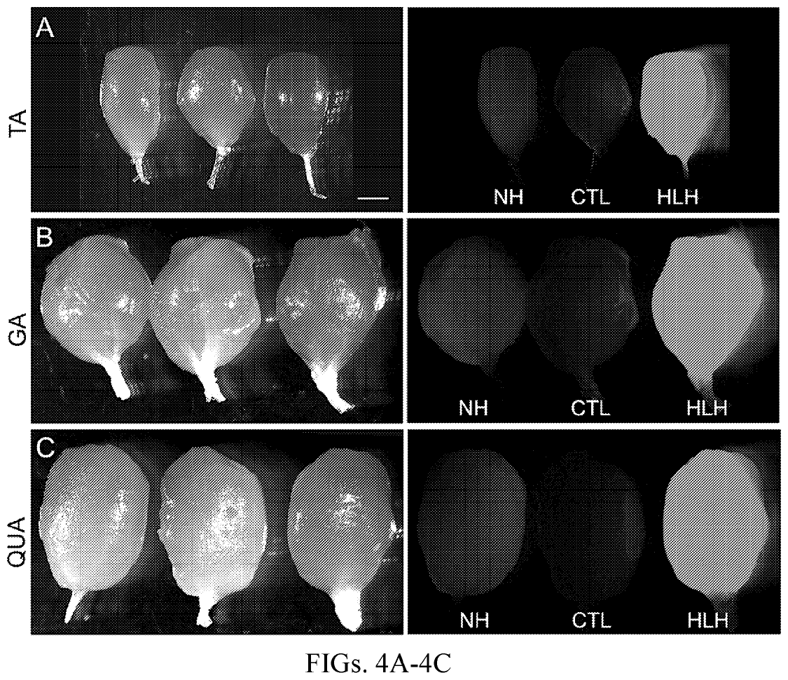

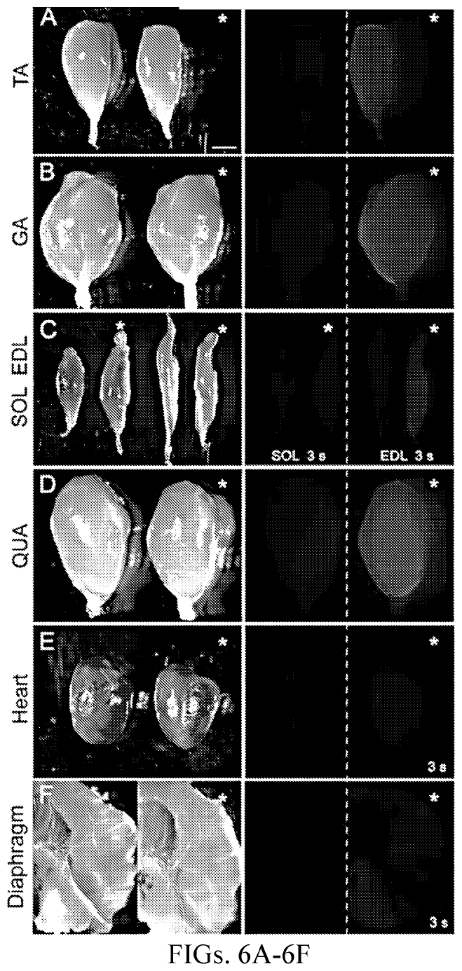

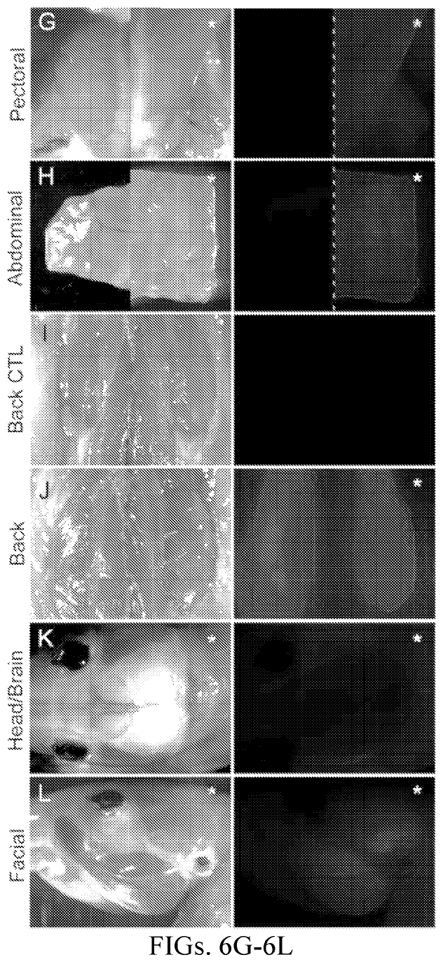

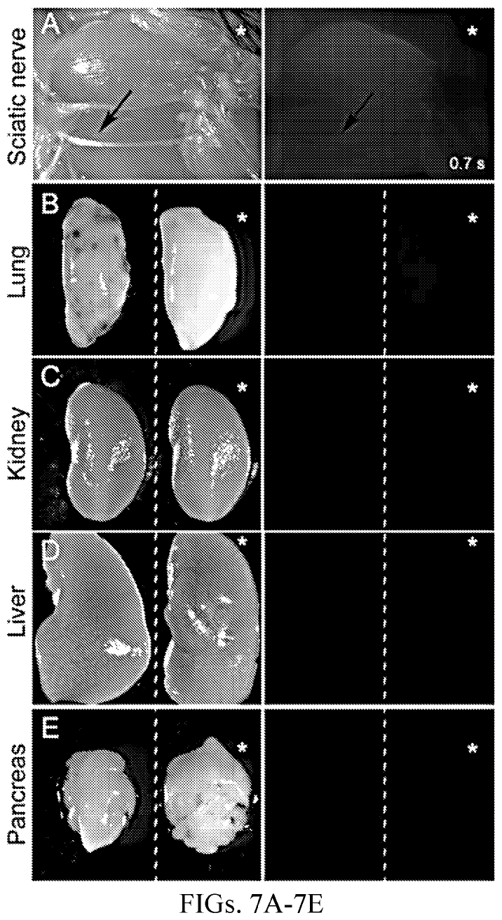

Mice injected with NH-mCherry in rAAVrh74 showed strong expression in tibialis anterior (TA), gastrocnemius (GA), and quadriceps (QUA), as well as pectoral, abdominal, back, and facial muscles (FIGS. 3-4, 6). Expression was virtually undetectable in soleus (SOL), EDL, diaphragm, and heart (FIGS. 3-4), even at a higher exposure (FIG. 6), and in non-muscle tissues (FIG. 7). This recapitulates the expression pattern we observed with this cassette delivered in AAV9 virions (Himeda et al. (2021) Mol Ther Methods Clin Dev. 20: p 298-311), confirming that low activity in certain skeletal muscles and virtually undetectable activity in the heart is intrinsic to NH and not due to differences in vector transducibility.

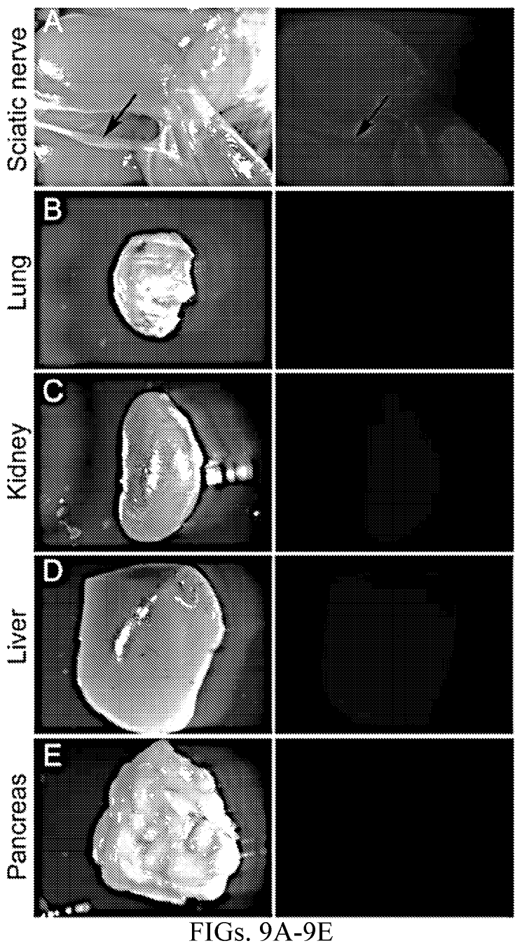

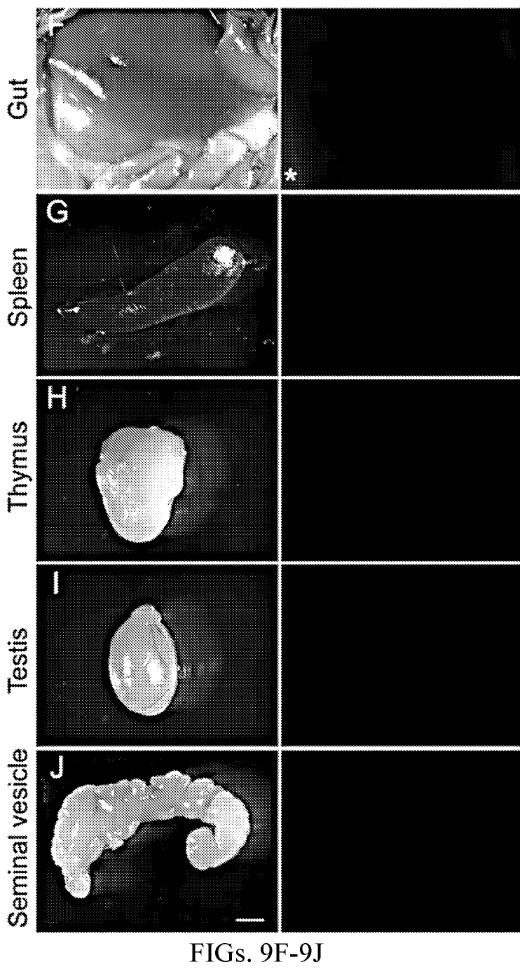

Restoration of the left E-box in HLH (FIG. 1) dramatically improved reporter expression in all fast-twitch muscles, provided low expression in the heart, and most importantly allowed for moderate expression in SOL, EDL, and diaphragm (FIGS. 3-4, 8). The total flux measurement showed 8.6, 12.3, 20, and 36.5× higher mCherry expression with HLH vs NH in QUA, GA, TA, and EDL muscles (FIG. 3A), and their high expression turned muscles pink even under visible light (FIG. 4). By contrast, low mCherry signals in the heart despite its size and higher AAV transducibility than skeletal muscles indicate that this cassette has low activity in the heart. To eliminate the influence of tissue size on signal measurement, we used max radiance data from the in vitro optical imaging analysis to compare mCherry signals in skeletal muscles and heart (FIG. 3B), and identified high to low HLH activity as follows: TA=GA=QUA (high: ˜2.5×)>EDL=abdomen (moderate: 1×)>heart=SOL (low: ˜0.3×). Since max radiance of the HLH mCherry signal in abdominal muscle was 2.5×10¹¹ and 5.12× more than NH (4.96×10¹⁰), comparative HLH activities over NH (abdominal activity=1) are TA=GA=QUA (high: ˜12.8×)>EDL=abdomen (moderate: 5.1×)>heart=SOL (low: ˜1.5×). We conclude that HLH displays extremely high activity in most skeletal muscles, with no detectable expression in non-muscle tissues (FIG. 9).

### Example 3: Selected Discussion

The studies presented herein disclose two highly minimized, muscle-specific cassettes with utility for different gene therapy strategies across a wide range of muscle disorders. Due to their small size, both cassettes can be used for diverse strategies, including gene replacement, exon skipping, and CRISPR-based gene editing, activation, or inhibition approaches. Allowing for increased transgene size downstream of tissue-specific gene regulation is not trivial, since clinically relevant gene therapies utilizing rAAV require therapeutic cassettes contained within single vectors (Hareendran et al. (2013) Rev Med Virol. 23 (6): p 399-413). Eliminating the need for two or more vectors is critical for increasing the efficiency of delivery, lowering the high cost of therapy, and reducing the immunotoxicity associated with high viral doses. This is a particular challenge for some CRISPR-based approaches, which often utilize multiple vectors to accommodate all therapeutic components, and for gene replacement approaches to DMD, in which the ability to increase cargo size by even a few hundred bp can be critical to efficacy. Importantly, the minimized regulatory cassettes of the current invention enable a single-vector platform. As a non-limiting example, at 378 bp, they can be accommodated into all-in-one therapeutic cassettes containing dSaCas9 fused to a minimized effector domain and all sgRNA components within the 4.4-kb packaging limit for rAAV vectors. When combined with the engineered mini-Cas9 proteins or recently described smaller Cas9 orthologs, which could allow for multiple sgRNA cassettes or larger effector domain fusions, the potential therapeutic applications are even greater.

The NH cassette of the current invention provides high-level expression in most fast-twitch skeletal muscles and essentially no expression in heart. Thus, it is ideally suited to the treatment of skeletal myopathies where cardiac muscle is unaffected, including but not limited to facioscapulohumeral muscular dystrophy (FSHD), X-linked myotubular myopathy (XLMTM), central core myopathy, inclusion body myositis, most cases of nemaline myopathy, distal myopathy, centronuclear myopathy, and oculopharyngeal muscular dystrophy, and certain subtypes of limb-girdle muscular dystrophies (LGMDs) where patients show no cardiac involvement. Examples of the latter include LGMD2A/R1 (calpainopathy), one of the most common LGMD subtypes, and most dysferlinopathies.

By contrast to the NH cassette, the HLH cassette provides moderate to very high-level expression across all skeletal muscles in conjunction with low-level expression in heart, making it well-suited to the treatment of disorders that affect all striated muscles, but where high expression of the therapeutic product in cardiac muscle could be detrimental. These include but are not limited to Duchenne muscular dystrophy (DMD) and several subtypes of LGMD and EDMD. For these diseases, effective treatment requires some therapeutic expression in cardiac muscle, but the results from clinical trials suggest that lowering expression levels in heart will prevent myocarditis from occurring. Since AAV capsids that are highly tropic for skeletal muscle are also highly tropic for cardiac muscle, the best way to selectively reduce transgene expression in the heart is via transcriptional regulation. Thus, the HLH cassette of the current invention may provide the optimal regulation for therapeutic transgenes in these indications. In addition, since HLH was highly active in all skeletal muscles (including soleus and EDL, where NH was nearly inactive), the HLH cassette would be well-suited for approaches where high skeletal muscle transgene expression is desirable and low cardiac transgene expression is well-tolerated. In FSHD, for example, the soleus is often severely affected and would be a desirable target muscle for therapeutic delivery. Thus, despite the lack of clinical cardiac pathology in FSHD, the HLH cassette may be a better overall therapeutic choice for this and potentially other myopathies.

The studies disclosed herein describe two novel variations of the CK8 regulatory cassette widely used in gene therapy approaches for muscular dystrophies. These minimized cassettes provide significantly increased space for transgene cargo while maintaining high activity and specificity for skeletal muscle combined with no (NH) or low (HLH) activity in the heart. Importantly, neither cassette has any detectable activity in non-muscle tissues regardless of viral transduction levels. Without wishing to be bound by theory, these new cassettes will be a valuable tool for increasing the safety of gene therapy approaches for a number of myopathies.

### Enumerated Embodiments

The following enumerated embodiments are provided, the numbering of which is not to be construed as designating levels of importance.

Embodiment 1 provides a polynucleotide encoding a muscle-specific regulatory cassette comprising at least three modified enhancer sequences upstream and operably linked to a promoter,
wherein the three modified enhancer sequences comprise a Trex sequence, an AT-rich sequence, a right E-box sequence, and a MEF2 sequence,
wherein the right E-box sequence is repeated at least once, and wherein the promoter is a −80 to +50 promoter containing a consensus Inr sequence.

Embodiment 2 provides the polynucleotide of embodiment 1, wherein the cassette has high expression activity in skeletal muscle tissue.

Embodiment 3 provides the polynucleotide of embodiment 2, wherein the skeletal muscle tissue is fast-twitch muscle tissue.

Embodiment 4 provides the polynucleotide of embodiment 1, wherein the cassette has very low expression in cardiac tissue as compared to skeletal muscle tissue.

Embodiment 5 provides the polynucleotide of embodiment 1, wherein the cassette is encoded by a nucleic acid comprising the sequence set forth in SEQ ID NO: 1.

Embodiment 6 provides the polynucleotide of embodiment 1, wherein the cassette is encoded by a nucleic acid comprising at least 80%, at least 85%, at least 90%, at least 95%, at least 98%, and at least 99% sequence identity to the sequence set forth in SEQ ID NO: 1.

Embodiment 7 provides a polynucleotide encoding a muscle-specific regulatory cassette comprising at least three modified enhancer sequences upstream and operably linked to a promoter,
wherein the at least three modified enhancer sequences comprise a Trex sequence, an AT-rich sequence, a left E-box sequence, a right E-box sequence, and a MEF2 sequence,
wherein the promoter is a −80 to +50 promoter containing a consensus Inr sequence.

Embodiment 8 provides the polynucleotide of embodiment 7, wherein the cassette has high expression activity in skeletal muscle tissue.

Embodiment 9 provides the polynucleotide of embodiment 7, wherein the cassette has low expression activity in cardiac tissue compared to skeletal muscle tissue.

Embodiment 10 provides the polynucleotide of embodiment 7, wherein the cassette is encoded by a nucleic acid comprising the sequence set forth in SEQ ID NO: 2.

Embodiment 11 provides the polynucleotide of embodiment 7, wherein the cassette is encoded by a nucleic acid comprising at least 80%, at least 85%, at least 90%, at least 95%, at least 98%, and at least 99% sequence identity to the sequence set forth in SEQ ID NO: 2.

Embodiment 12 provides an AAV vector comprising a muscle-specific regulatory cassette comprising the polynucleotide of any one of embodiments 1-6.

Embodiment 13 provides an AAV vector comprising a muscle-specific regulatory cassette comprising the polynucleotide of any one of embodiments 7-11.

Embodiment 14 provides the AAV vector of any one of embodiments 12-13, comprising an AAV capsid that is a serotype selected from the group consisting of AAV1, AAV2, AAV3, AAV3B, AAV4, AAV5, AAV6, AAV7, AAV8, AAV9, AAV12, AAV-B1, AAV-DJ, AAV-Retro, AAVrh8, AAVrh10, AAVrh25, Anc80L65, LK03, AAVrh18, rAAVrh74, AAVrh32.33, AAVrh39, AAVrh43, MyoAAV2, Oligo001, PHP-B, and Spark100.

Embodiment 15 provides the AAV vector of embodiment 14, wherein the capsid is an rAAVrh74 serotype.

Embodiment 16 provides the AAV vector of embodiment 14, wherein the capsid is an AAV9 serotype.

Embodiment 17 provides the AAV vector of embodiment 14, wherein the capsid is a MyoAAV2 serotype.

Embodiment 18 provides the AAV vector of embodiment 14, wherein the capsid has a specificity for muscle tissue.

Embodiment 19 provides a composition comprising AAV vector particles comprising the polynucleotide of any one of embodiments 1-11 and a pharmaceutically acceptable carrier or excipient.

Embodiment 20 provides a method of treating a genetic disease or disorder in a subject in need thereof, comprising administering to the subject an effective amount of an AAV vector comprising a muscle-specific regulatory cassette comprising at least three modified enhancer sequences upstream and operably linked to a promoter, thereby treating the genetic disease or disorder,
wherein the three modified enhancer sequences comprise a Trex sequence, an AT-rich sequence, a right E-box sequence, and a MEF2 sequence,
wherein the right E-box sequence is repeated at least once, and
wherein the promoter is a −80 to +50 promoter containing a consensus Inr sequence.

Embodiment 21 provides the method of embodiment 20, wherein the cassette is encoded by a nucleic acid comprising the sequence set forth in SEQ ID NO: 1.

Embodiment 22 provides a method of treating a genetic disease or disorder in a subject in need thereof, comprising administering to the subject an effective amount of an AAV vector comprising a polynucleotide encoding a muscle-specific regulatory cassette comprising at least three modified enhancer sequences upstream and operably linked to a promoter,
wherein the at least three modified enhancer sequences comprise a Trex sequence, an AT-rich sequence, a left E-box sequence, a right E-box sequence, and a MEF2 sequence,
wherein the promoter is a −80 to +50 promoter containing a consensus Inr sequence.

Embodiment 23 provides the method of embodiment 22, wherein the cassette is encoded by a nucleic acid comprising the sequence set forth in SEQ ID NO: 2.

Embodiment 24 provides the method of any one of embodiments 20-23, wherein the AAV vector comprises an AAV capsid that is a serotype selected from the group consisting of AAV1, AAV2, AAV3, AAV3B, AAV4, AAV5, AAV6, AAV7, AAV8, AAV9, AAV12, AAV-B1, AAV-DJ, AAV-Retro, AAVrh8, AAVrh10, AAVrh25, Anc80L65, LK03, AAVrh18, TAAVrh74, AAVrh32.33, AAVrh39, AAVrh43, MyoAAV2, Oligo001, PHP-B, and Spark 100.

Embodiment 25 provides the method of embodiment 24, wherein the capsid is an rAAVrh74 serotype.

Embodiment 25 provides the method of embodiment 24, wherein the capsid is an AAV9 serotype.

Embodiment 26 provides the method of embodiment 24, wherein the capsid is a MyoAAV2 serotype.

Embodiment 27 provides the method of any one of embodiments 20-23, wherein the AAV vector has a tropism for muscle tissue.

Embodiment 28 provides the method of any one of embodiments 20-26, wherein the genetic disease or disorder is a muscle-related genetic disease or disorder.

Embodiment 29 provides the method of embodiment 28, wherein the muscle-related genetic disease or disorder is selected from the list consisting of facioscapulohumeral muscular dystrophy (FSHD), X-linked myotubular myopathy (XLMTM), central core myopathy, inclusion body myositis, nemaline myopathy, distal myopathy, centronuclear myopathy, oculopharyngeal muscular dystrophy, a dysferlinopathy, a limb-girdle muscular dystrophy (LGMD), and Duchenne muscular dystrophy (DMD).

Embodiment 30 provides a method of introducing a transgene into a target cell, comprising contacting the cell with a polynucleotide encoding a muscle-specific regulatory cassette comprising at least three modified enhancer sequences upstream and operably linked to a promoter,
wherein the three modified enhancer sequences comprise a Trex sequence, an AT-rich sequence, a right E-box sequence, and a MEF2 sequence,
wherein the right E-box sequence is repeated at least once, and
wherein the promoter is a −80 to +50 promoter containing a consensus Inr sequence.

Embodiment 31 provides the method of embodiment 30, wherein the cassette has high expression activity in skeletal muscle tissue.

Embodiment 32 provides the method of embodiment 30, wherein the skeletal muscle tissue is fast-twitch muscle tissue.

Embodiment 33 provides the method of embodiment 30, wherein the cassette has very low expression in cardiac tissue as compared to skeletal muscle tissue.

Embodiment 34 provides the method of embodiment 30, wherein the cassette is encoded by a nucleic acid comprising the sequence set forth in SEQ ID NO: 1.

Embodiment 35 provides the method of embodiment 30, wherein the cassette is encoded by a nucleic acid comprising at least 80%, at least 85%, at least 90%, at least 95%, at least 98%, and at least 99% sequence identity to the sequence set forth in SEQ ID NO: 1.

Embodiment 36 provides a method of introducing a transgene into a target cell, comprising contacting the cell with a polynucleotide encoding a muscle-specific regulatory cassette comprising at least three modified enhancer sequences upstream and operably linked to a promoter,
wherein the at least three modified enhancer sequences comprise a Trex sequence, an AT-rich sequence, a left E-box sequence, a right E-box sequence, and a MEF2 sequence,
wherein the promoter is a −80 to +50 promoter containing a consensus Inr sequence.

Embodiment 37 provides the method of embodiment 36, wherein the cassette has high expression activity in skeletal muscle tissue.

Embodiment 38 provides the method of embodiment 36, wherein the cassette has low expression activity in cardiac tissue compared to skeletal muscle tissue.

Embodiment 39 provides the method of embodiment 36, wherein the cassette is encoded by a nucleic acid comprising the sequence set forth in SEQ ID NO: 2.

Embodiment 40 provides the method of embodiment 36, wherein the cassette is encoded by a nucleic acid comprising at least 80%, at least 85%, at least 90%, at least 95%, at least 98%, and at least 99% sequence identity to the sequence set forth in SEQ ID NO: 2.

### Other Embodiments

The recitation of a listing of elements in any definition of a variable herein includes definitions of that variable as any single element or combination (or subcombination) of listed elements. The recitation of an embodiment herein includes that embodiment as any single embodiment or in combination with any other embodiment or portions thereof.

The disclosures of each and every patent, patent application, and publication cited herein are hereby incorporated herein by reference in their entirety. While this invention has been disclosed with reference to specific embodiments, it is apparent that other embodiments and variations of this invention may be devised by others skilled in the art without departing from the true spirit and scope of the invention. The appended claims are intended to be construed to include all such embodiments and equivalent variations.

What is claimed is:

1. A polynucleotide encoding a muscle-specific regulatory cassette comprising at least three modified enhancer sequences upstream and operably linked to a promoter,
wherein the three modified enhancer sequences comprise a Trex sequence, an AT-rich sequence, a right E-box sequence, and a MEF2 sequence,
wherein the right E-box sequence is repeated at least once, and
wherein the promoter is a −80 to +50 promoter containing a consensus Inr sequence.

2. The polynucleotide of claim 1, wherein the cassette has high expression activity in skeletal muscle tissue.

3. The polynucleotide of claim 2, wherein the skeletal muscle tissue is fast-twitch muscle tissue.

4. The polynucleotide of claim 1, wherein the cassette has very low expression in cardiac tissue as compared to skeletal muscle tissue.

5. The polynucleotide of claim 1, wherein the cassette is encoded by a nucleic acid comprising the sequence set forth in SEQ ID NO: 1.

6. The polynucleotide of claim 1, wherein the cassette is encoded by a nucleic acid comprising at least 80%, at least 85%, at least 90%, at least 95%, at least 98%, and at least 99% sequence identity to the sequence set forth in SEQ ID NO: 1.

7. A polynucleotide encoding a muscle-specific regulatory cassette comprising at least three modified enhancer sequences upstream and operably linked to a promoter,
wherein the at least three modified enhancer sequences comprise a Trex sequence, an AT-rich sequence, a left E-box sequence, a right E-box sequence, and a MEF2 sequence,
wherein the promoter is a −80 to +50 promoter containing a consensus Inr sequence.

8. The polynucleotide of claim 7, wherein the cassette has high expression activity in skeletal muscle tissue.

9. The polynucleotide of claim 7, wherein the cassette has low expression activity in cardiac tissue compared to skeletal muscle tissue.

10. The polynucleotide of claim 7, wherein the cassette is encoded by a nucleic acid comprising the sequence set forth in SEQ ID NO: 2.

11. The polynucleotide of claim 7, wherein the cassette is encoded by a nucleic acid comprising at least 80%, at least 85%, at least 90%, at least 95%, at least 98%, and at least 99% sequence identity to the sequence set forth in SEQ ID NO: 2.

12. An AAV vector comprising a muscle-specific regulatory cassette comprising the polynucleotide of any one of claims 1-6.

13. An AAV vector comprising a muscle-specific regulatory cassette comprising the polynucleotide of any one of claims 7-11.

14. The AAV vector of any one of claims 12-13, comprising an AAV capsid that is a serotype selected from the group consisting of AAV1, AAV2, AAV3, AAV3B, AAV4, AAV5, AAV6, AAV7, AAV8, AAV9, AAV12, AAV-B1, AAV-DJ, AAV-Retro, AAVrh8, AAVrh10, AAVrh25, Anc80L65, LK03, AAVrh18, rAAVrh74, AAVrh32.33, AAVrh39, AAVrh43, MyoAAV2, Oligo001, PHP-B, and Spark100.

15. The AAV vector of claim 14, wherein the capsid is an rAAVrh74 serotype.

16. The AAV vector of claim 14, wherein the capsid is an AAV9 serotype.

17. The AAV vector of claim 14, wherein the capsid is a MyoAAV2 serotype.

18. The AAV vector of claim 14, wherein the capsid has a specificity for muscle tissue.

19. A composition comprising AAV vector particles comprising the polynucleotide of any one of claims 1-11 and a pharmaceutically acceptable carrier or excipient.

20. A method of treating a genetic disease or disorder in a subject in need thereof, comprising administering to the subject an effective amount of an AAV vector comprising a muscle-specific regulatory cassette comprising at least three modified enhancer sequences upstream and operably linked to a promoter, thereby treating the genetic disease or disorder,
wherein the three modified enhancer sequences comprise a Trex sequence, an AT-rich sequence, a right E-box sequence, and a MEF2 sequence,
wherein the right E-box sequence is repeated at least once, and
wherein the promoter is a −80 to +50 promoter containing a consensus Inr sequence.

21. The method of claim 20, wherein the cassette is encoded by a nucleic acid comprising the sequence set forth in SEQ ID NO: 1.

22. A method of treating a genetic disease or disorder in a subject in need thereof, comprising administering to the subject an effective amount of an AAV vector comprising a muscle-specific regulatory cassette comprising at least three modified enhancer sequences upstream and operably linked to a promoter,
wherein the at least three modified enhancer sequences comprise a Trex sequence, an AT-rich sequence, a left E-box sequence, a right E-box sequence, and a MEF2 sequence,
wherein the promoter is a −80 to +50 promoter containing a consensus Inr sequence.

23. The method of claim 22, wherein the cassette is encoded by a nucleic acid comprising the sequence set forth in SEQ ID NO: 2.

24. The method of any one of claims 20-23, wherein the AAV vector comprises an AAV capsid that is a serotype selected from the group consisting of AAV1, AAV2, AAV3, AAV3B, AAV4, AAV5, AAV6, AAV7, AAV8, AAV9, AAV12, AAV-B1, AAV-DJ, AAV-Retro, AAVrh8, AAVrh10, AAVrh25, Anc80L65, LK03, AAVrh18, rAAVrh74, AAVrh32.33, AAVrh39, AAVrh43, MyoAAV2, Oligo001, PHP-B, and Spark100.

25. The method of claim 24, wherein the capsid is an rAAVrh74 serotype.

26. The method of claim 24, wherein the capsid is an AAV9 serotype.

27. The method of claim 24, wherein the capsid is a MyoAAV2 serotype.

28. The method of any one of claims 20-27, wherein the AAV vector has a specificity for muscle tissue.

29. The method of any one of claims 20-27, wherein the genetic disease or disorder is a muscle-related genetic disease or disorder.

30. The method of claim 29, wherein the muscle-related genetic disease or disorder is selected from the list consisting of facioscapulohumeral muscular dystrophy (FSHD), X-linked myotubular myopathy (XLMTM), central core myopathy, inclusion body myositis, nemaline myopathy, distal myopathy, centronuclear myopathy, oculopharyngeal muscular dystrophy, a dysferlinopathy, a limb-girdle muscular dystrophy (LGMD), and Duchenne muscular dystrophy (DMD).

31. A method of introducing a transgene into a target cell, comprising contacting the cell with a polynucleotide encoding a muscle-specific regulatory cassette comprising at least three modified enhancer sequences upstream and operably linked to a promoter,
wherein the three modified enhancer sequences comprise a Trex sequence, an AT-rich sequence, a right E-box sequence, and a MEF2 sequence,
wherein the right E-box sequence is repeated at least once, and
wherein the promoter is a −80 to +50 promoter containing a consensus Inr sequence.

32. The method of claim 31, wherein the cassette has high expression activity in skeletal muscle tissue.

33. The method of claim 32, wherein the skeletal muscle tissue is fast-twitch muscle tissue.

34. The method of claim 31, wherein the cassette has very low expression in cardiac tissue as compared to skeletal muscle tissue.

35. The method of claim 31, wherein the cassette is encoded by a nucleic acid comprising the sequence set forth in SEQ ID NO: 1.

36. The method of claim 31, wherein the cassette is encoded by a nucleic acid comprising at least 80%, at least 85%, at least 90%, at least 95%, at least 98%, and at least 99% sequence identity to the sequence set forth in SEQ ID NO: 1.

37. A method of introducing a transgene into a target cell, comprising contacting the cell with a polynucleotide encoding a muscle-specific regulatory cassette comprising at least three modified enhancer sequences upstream and operably linked to a promoter,
wherein the at least three modified enhancer sequences comprise a Trex sequence, an AT-rich sequence, a left E-box sequence, a right E-box sequence, and a MEF2 sequence,
wherein the promoter is a −80 to +50 promoter containing a consensus Inr sequence.

38. The method of claim 37, wherein the cassette has high expression activity in skeletal muscle tissue.

39. The method of claim 37, wherein the cassette has low expression activity in cardiac tissue compared to skeletal muscle tissue.

40. The method of claim 37, wherein the cassette is encoded by a nucleic acid comprising the sequence set forth in SEQ ID NO: 2.

41. The method of claim 37, wherein the cassette is encoded by a nucleic acid comprising at least 80%, at least 85%, at least 90%, at least 95%, at least 98%, and at least 99% sequence identity to the sequence set forth in SEQ ID NO: 2.


SEQUENCE LISTING (2 sequences)

SEQ ID NO: 1 - DNA, 378 bp, synthetic construct
  source 1..378: mol_type = other DNA
ggacacccgagacgcccggttataattaaccccaacacctgcccccccccccaacacctgcccgacctctaaaaataactcctcggacacccgagacgcccggttataattaaccccaacacctgcccccccccccaacacctgcccgacctctaaaaataactcctcggacacccgagacgcccggttataattaaccccaacacctgcccccccccccaacacctgcccgacctctaaaaataacccctccctggggacaacccctcccagccaatagcacagcctaggtccccctatataaggccacggctgctggcccttcctcattctcagtgtcacctccaggatacagacagccccccttcagcccagccc

SEQ ID NO: 2 - DNA, 379 bp, synthetic construct
  source 1..379: mol_type = other DNA
ggacacccgagacgcccggttataattaaccaggacacgtggcgaccccccccaacacctgcccgacctctaaaaataactcctggacacccgagacgcccggttataattaaccaggacacgtggcgaccccccccaacacctgcccgacctctaaaaataactcctggacacccgagacgcccggttataattaaccaggacacgtggcgaccccccccaacacctgcccgacctctaaaaataacccctccctggggacaacccctcccagccaatagcacagcctaggtccccctatataaggccacggctgctggcccttcctcattctcagtgtcacctccaggatacagacagccccccttcagcccagccc